(19) United States
(12) Patent Application Publication
(10) Pub. No.: US 2022/0211792 A1
(43) Pub. Date: Jul. 7, 2022

(54) COMPOSITION CONTAINING NATURAL EXTRACTS FOR ENHANCEMENT OF INNATE IMMUNITY OR ANTIVIRAL USE AGAINST INFLUENZA VIRUS OR CORONA VIRUS

(71) Applicant: OKCHUNDANG CO., LTD., Daegu (KR)

(72) Inventors: Chul Jong JUNG, Daegu (KR); Seok Man PARK, Daegu (KR); Ryong KONG, Daegu (KR); Yoeng Eun YU, Daegu (KR)

(21) Appl. No.: 17/564,459

(22) Filed: Dec. 29, 2021

(30) Foreign Application Priority Data
Jan. 4, 2021 (KR) 10-2021-0000525
Nov. 17, 2021 (KR) 10-2021-0158498

(51) Int. Cl. A61K 36/64; A61K 36/344; A61K 36/258; A61K 36/346; A61K 36/076; A61K 36/734; A61K 36/481; A61P 37/04; A61P 31/12

(52) U.S. Cl. CPC A61K 36/64; A61K 36/344; A61K 36/258; A61K 36/346; A61P 31/12; A61K 36/734; A61K 36/481; A61P 37/04; A61K 36/076

(57) ABSTRACT

Disclosed is a composition containing natural extracts for enhancement of innate immunity or antiviral use against influenza virus (PR8) or corona virus, wherein the composition promotes interferon secretion in the innate defense immune system to induce protection from the antiviral infection and thus can be effectively used for antiviral use.

### SEQUENCE LISTING

The instant application contains a Sequence Listing which has been submitted electronically in ASCII format and is hereby incorporated by reference in its entirety. Said ASCII copy, created on Dec. 28, 2021 is named 50413-226001_Sequence_Listing_12_28_21_ST25 and is 2,690 bytes in size.

### BACKGROUND OF THE INVENTION

### Field of the Invention

The present disclosure relates to a composition containing natural extracts for enhancement of innate immunity, antiviral use against influenza virus (PR8) or corona virus, or prevention, treatment, or alleviation of a viral infection and, more specifically, to a composition for enhancement of innate immunity, antiviral use, or prevention, treatment, or alleviation of a viral infection, wherein the composition promotes interferon secretion in the innate defense immune system to induce protection from the antiviral infection and thus can be effectively used for antiviral use.

The present disclosure relates to a method for enhancement of innate immunity, antiviral use, or alleviation, prevention, or treatment of a viral infection, the method including administering to a subject a composition containing a first complex extract of Codonopsis and Rehmannia.

The present disclosure relates to a method for enhancement of innate immunity, antiviral use, or alleviation, prevention, or treatment of a viral infection, the method including administering to a subject a composition containing a second complex extract of two or more selected from the group consisting of Codonopsis, Rehmannia, Ginseng, Platycodon, Poria, Hawthorn fruit, and Astragalus.

### DESCRIPTION OF THE PRIOR ART

Immunity refers to a series of biological defense responses that occur to prevent substances other than the body's own components from breaking homeostasis of the body or threatening the body itself. All immune responses including the inflammatory response activated due to the influx of pathogens into the body return to a normal state when the pathogens are removed, but the immune responses are remarkably high or low compared with normal persons when there is difficulty in immune regulation.

The deficiency or deterioration in immune functions is called immunological incompetence, and in this state, the immune responses are not properly activated and thus do not respond to foreign substances (bacteria, viruses, etc.) entering the body, causing infections.

Conversely, immune hypersensitivity reactions are some immune responses that are exaggerated to result in an imbalance of the immune system, causing allergic responses. In the immune system that is not properly regulated and is imbalanced, immunoregulation disorders are caused by cytokine imbalance, T/B cell proliferation imbalance, and antioxidant nutrient depletion in the body, and thus the secretion of inflammatory substances may increase to result in damage to cells and tissues and inflammation may be aggravated by the failure to cope with the influx of pathogens. Therefore, the immune response of the human body must be balanced and normally regulated in order to maintain health, and thus the immunoregulatory ability is important for the prevention and treatment of diseases.

Currently, antibody drugs account for the largest proportion of medicines used for autoimmune diseases. Antibody drugs treat immune diseases by using antibodies that can specifically bind to antigens, and representative antibody drugs are Amgen's Enbrel, Johnson & Johnson's Remicade, and the like. These are medicines that are mainly used for immune diseases such as rheumatoid arthritis and are medicines that are currently receiving attention as immunotherapeutic agents that regulate excessive immune responses by inhibiting the cytokine TNF-α in the cellular mechanism. However, the overdose of these drugs as medicines inhibiting the immune response may cause symptoms, such as infectious diseases, allergic reactions, itching, injection site erythema (including bleeding, bruising, erythema, pain, and swelling), dyspnea, and fever.

In addition, the decline in immunity due to the failure of immunoregulation may be a cause of an infection with virus or the like. The virus means a toxic substance in Latin, and refers to a group of infectious pathogenic particles that pass through a bacteriological filter bed (0.22 um). Viruses may be classified into bacteriophages, plant viruses, and animal viruses according to the type of host cell, and may be classified into DNA viruses and RNA viruses according to the type of nucleic acid. Recently, various viral diseases, such as COVID-19, swine flu, AI, and foot-and-mouth disease, have caused great social problems, and therefore, the concerns about effective countermeasures for viral diseases are arousing great social interest.

Although one of the best ways to prevent viral diseases is vaccination, the problems of vaccine effectiveness due to the generation of many viral serotypes (subtypes) are being raised as an important issue in the viral diseases. The development and distribution of inhibitors for virus prevention that can solve the problems of these vaccines is important, and to this end, boosting the immunity of individual animals by stimulating the in vivo innate immune system, which is the initial defense system against viruses, can be another solution.

In recent years, the field of innate immunity induced by interferons in the in vivo innate defense immune system has been receiving attention, and the innate immunity is one of the defense mechanisms of innate immunity, wherein the secretion of immune factors (inflammatory cytokines) is induced to cause an inflammatory response in the body, thereby defending against pathogens. Therefore, the induction of inflammatory response at an appropriate level is considered to be effective in the prevention of infectious diseases caused by pathogens such as viruses, and this is the reason why research on innate immunity enhancing agents is necessary. In particular, the utilization of herbal extracts or plant extracts, which have little toxicity and side effects as raw materials for preparations, enables the development of preparations that can also be used as food or health functional food.

### SUMMARY OF THE INVENTION

The present disclosure has been made in order to solve the above-mentioned problems in the prior art, and the present inventors developed a composition for enhancement of innate immunity, antiviral use, and prevention and treatment of a viral infection, by using natural products, which have few side effects as disadvantages of conventionally used medicines and are usable as food, and confirmed that the extract to be developed had an excellent effect on innate immunity enhancement and antiviral action.

A first object of the present disclosure is to provide a composition for enhancement of innate immunity, antiviral use, and prevention and treatment of a viral infection, the composition containing at least one selected from Codonopsis, Rehmannia, Ginseng, Platycodon, Poria, Hawthorn fruit, and Astragalus, and to provide a health functional food using the same.

Accordingly, an aspect of the present disclosure is to provide a pharmaceutical composition for enhancement of innate immunity, antiviral use, or prevention or treatment of a viral infection, the pharmaceutical composition containing a complex extract of two or more selected from the group consisting of Codonopsis, Rehmannia, Ginseng, Platycodon, Poria, Hawthorn fruit, and Astragalus.

Another aspect of the present disclosure is to provide a health functional food for enhancement of innate immunity, antiviral use, or prevention or treatment of a viral infection, the health functional food containing a complex extract of two or more selected from the group consisting of Codonopsis, Rehmannia, Ginseng, Platycodon, Poria, Hawthorn fruit, and Astragalus.

Still another aspect of the present disclosure is to provide use of a complex extract of two or more selected from the group consisting of Codonopsis, Rehmannia, Ginseng, Platycodon, Poria, Hawthorn fruit, and Astragalus, for enhancement of innate immunity, antiviral use, or prevention or treatment of a viral infection.

Still another aspect of the present disclosure is to provide a method for enhancement of innate immunity, antiviral use, or alleviation, prevention, or treatment of a viral infection, the method including administering to a subject a composition containing a first complex extract of Codonopsis and Rehmannia.

Still another aspect of the present disclosure is to provide a method for enhancement of innate immunity, antiviral use, or alleviation, prevention, or treatment of a viral infection, the method including administering to a subject a composition containing a second complex extract of two or more selected from the group consisting of Codonopsis, Rehmannia, Ginseng, Platycodon, Poria, Hawthorn fruit, and Astragalus.

In order to achieve the first object, the present disclosure provides a composition for enhancement of innate immunity, antiviral use, or alleviation, prevention, or treatment of a viral infection, the composition may contain as an active ingredient a complex extract of two or more selected from the group consisting of Codonopsis, Rehmannia, Ginseng, Platycodon, Poria, Hawthorn fruit, and Astragalus. Furthermore, the present disclosure provides a health functional food for enhancement of innate immunity, antiviral use, or prevention or treatment of a viral infection, the health functional food containing as an active ingredient a complex extract of two or more selected from the group consisting of Codonopsis, Rehmannia, Ginseng, Platycodon, Poria, Hawthorn fruit, and Astragalus.

Hereinafter, the present disclosure will be described in more detail.

In accordance with an aspect of the present disclosure, there is provided a pharmaceutical composition for enhancement of innate immunity, antiviral use, or prevention or treatment of a viral infection, the pharmaceutical composition containing a first complex extract of Codonopsis and Rehmannia.

The first complex extract may contain 5.9-8.9 wt % of Codonopsis and 74.1-94.1 wt % of Rehmannia and, preferably 6.7-8.1 wt % of Codonopsis and 83.3-93.3 wt % of Rehmannia, but is not limited thereto.

In the present disclosure, the first complex extract may further contain at least one selected from the group consisting of Ginseng and Platycodon.

The first complex extract may contain 5.4-8.2 wt % of Codonopsis, 6.4-9.6 wt % of Ginseng, and 68.2-88.2 wt % of Rehmannia, and preferably 6.1-7.5 wt % of Codonopsis, 7.2-8.8 wt % of Ginseng, and 76.7-86.7 wt % of Rehmannia, but is not limited thereto.

The first complex extract may contain 5.0-7.4 wt % of Codonopsis, 62.0-82.0 wt % of Rehmannia, and 13.0-19.6 wt % of Platycodon, and preferably 5.6-6.8 wt % of Codonopsis, 69.8-79.8 wt % of Rehmannia, and 14.7-17.9 wt % of Platycodon, but is not limited thereto.

In the present disclosure, the first complex extract may further contain at least one selected from the group consisting of Poria and Hawthorn fruit.

The first complex extract may contain 4.0-6.0 wt % of Codonopsis, 4.7-7.1 wt % of Ginseng, 50.2-70.2 wt % of Rehmannia, 9.7-14.5 wt % of Poria, 0.9-1.3 wt % of Hawthorn fruit, and 10.6-15.8 wt % of Platycodon, and preferably, 4.5-5.5 wt % of Codonopsis, 5.3-6.5 wt % of Ginseng, 56.4-66.4 wt % of Rehmannia, 10.9-13.3 wt % of Poria, 1.0-1.2 wt % of Hawthorn fruit, and 11.9-14.5 wt % of Platycodon, but is not limited thereto.

In accordance with another aspect of the present disclosure, there is provided a health functional food for enhancement of innate immunity, antiviral use, or prevention or treatment of a viral infection, the health functional food containing a first complex extract of Codonopsis and Rehmannia.

The first complex extract may contain 5.9-8.9 wt % of Codonopsis and 74.1-94.1 wt % of Rehmannia and, preferably 6.7-8.1 wt % of Codonopsis and 83.3-93.3 wt % of Rehmannia.

In the present disclosure, the first complex extract may further contain at least one selected from the group consisting of Ginseng and Platycodon.

The first complex extract may contain 5.4-8.2 wt % of Codonopsis, 6.4-9.6 wt % of Ginseng, and 68.2-88.2 wt % of Rehmannia, and preferably 6.1-7.5 wt % of Codonopsis, 7.2-8.8 wt % of Ginseng, and 76.7-86.7 wt % of Rehmannia, but is not limited thereto.

The first complex extract may contain 5.0-7.4 wt % of Codonopsis, 62.0-82.0 wt % of Rehmannia, and 13.0-19.6 wt % of Platycodon, and preferably 5.6-6.8 wt % of Codonopsis, 69.8-79.8 wt % of Rehmannia, and 14.7-17.9 wt % of Platycodon, but is not limited thereto.

In the present disclosure, the first complex extract may further contain at least one selected from the group consisting of Poria and Hawthorn fruit.

The first complex extract may contain 4.0-6.0 wt % of Codonopsis, 4.7-7.1 wt % of Ginseng, 50.2-70.2 wt % of Rehmannia, 9.7-14.5 wt % of Poria, 0.9-1.3 wt % of Hawthorn fruit, and 10.6-15.8 wt % of Platycodon, and preferably, 4.5-5.5 wt % of Codonopsis, 5.3-6.5 wt % of Ginseng, 56.4-66.4 wt % of Rehmannia, 10.9-13.3 wt % of Poria, 1.0-1.2 wt % of Hawthorn fruit, and 11.9-14.5 wt % of Platycodon, but is not limited thereto.

In accordance with still another aspect of the present disclosure, there is provided a pharmaceutical composition for enhancement of innate immunity, antiviral use, or prevention or treatment of a viral infection, the pharmaceutical composition containing a second complex extract of two or more selected from the group consisting of Codonopsis, Rehmannia, Ginseng, Platycodon, Poria, Hawthorn fruit, and Astragalus.

The second complex extract may contain 1.25-5.0 wt % of Codonopsis, 12.50-50.0 wt % of Rehmannia, 1.25-5.00 wt % of Ginseng, 6.85-27.00 wt % of Platycodon, 2.50-10.00 wt % of Poria, 0.65-2.50 wt % of Hawthorn fruit, and 25.0-80.0 wt % of Astragalus, and preferably, 2.00-3.00 wt % of Codonopsis, 20.00-30.00 wt % of Rehmannia, 2.00-3.00 wt % of Ginseng, 10.00-20.00 wt % of Platycodon, 3.00-8.00 wt % of Poria, 1.00-1.50 wt % of Hawthorn fruit, and 40.0-60.0 wt % of Astragalus, but is not limited thereto.

The second complex extract may contain at least one compound selected from the group consisting of chlorogenic acid, ginsenoside Rg1, calycosin, ginsenoside Rb1, ginsenoside Rd, astragaloside II, astragaloside I, and polygalacin D, and for example, chlorogenic acid or ginsenoside Rd, but is not limited thereto.

As used herein, the term “Codonopsis” is Adenophora triphylla var. japonica Hara, Adenophora remotiflora (Siebold & Zucc.) Miq., Adenophora stricta Miq., Adenophora triphylla var. hirsuta Nakai, or a closely related congeneric plant, which is a perennial herb belonging to the family Campanulaceae. As for the appearance, the stems are about 50-100 cm in height straightly, and give a white liquid when broken. The leaves have a long ellipse, and four or five leaves are verticillate, with hairs on the stems and leaves. Several purplish flowers are versatile at the tip of the stem in July to October. The corolla has a bell shape and is 13-22 mm long. A style is divided into three, and is slightly longer than the corolla, and five stamens are dropped from the flower body. The stamen pole has a wide bottom and many hairs. Adenophora triphylla, Ginseng, Scrophularia buergeriana, Salvia miltiorrhiza, and Sophora flavescens are called five ginsengs, which have different forms but the same subjects to be treated. Codonopsis have pharmaceutical actions, such as antipyretic, antiviral, hemolytic, and cardiotonic actions.

In the present disclosure, Codonopsis may be Adenophora triphylla var. japonica Hara, Adenophora remotiflora (Siebold & Zucc.) Miq., or Adenophora stricta Miq., and the roots thereof may be selected for use, but is not limited thereto.

As used herein, the term “Ginseng” is Panax ginseng C. A Mey, which is a perennial herb belonging to the family Araliaceae, about 60 cm in height, one new stem growing straightly each year, one flower stalk connecting from one end of the stem, and 3 to 6 verticillate petioles. The leaves have long petioles, and a leaf blade being divided into 3-5, forming palmately compound leaves. Hairs are on leaf veins on a front surface of the leaf. In summer, a thin flower stalk comes out, and 4-40 small flowers having a light yellowish color come out from the tip of the flower stalk, hung in the umbel inflorescence. The flowers have five leaves and stamens and one pistil, with an inferior ovary. The fruit thereof is drupe, has an elliptic shape, and turns into bright red when being ripe. Ginseng has been called the best medicine for eternal longevity, giving vigor, and managing bodies.

In the present disclosure, Ginseng may be at least one selected from the group consisting of Panax ginseng, P. quiquefolius, P. notoginseng, P. japonicus, P. trifolium, P. pseudoginseng, and P. vietnamensis, and may be preferably P. ginseng or P. notoginseng, and may be at least one selected from the group consisting of fresh ginseng, tiny-sized ginseng, white ginseng, and red ginseng, and the roots thereof may be selected for use, but is not limited thereto.

As used herein, the term “Rehmannia” is Rehmannia glutinosa (Gaertner) Liboschitz or Rehmannia glutinosa for. hueichingensis (Chao et Schih) Hsia., which is a perennial herb belonging to the family Scrophulariaceae, about 30 cm in height. Leaves with an ellipse shape come out from roots, and purple-red flowers come out in June and July. In oriental medicine, Rehmanniae Radix is the raw root of the plant, Rehmanniae Radix Siccus is the root that has been dried, and Rehmanniae Radix Preparat is the root that has been steamed and dried. Rehmanniae Radix Preparat is used as a hematinic agent and for menstrual irregularity, a weak constitution, physical retardation of children, dementia, premature ejaculation, and impotence. Rehmanniae Radix is used for a weak constitution, hemoptysis, nasal bleeding, uterine bleeding, menstrual irregularity, and constipation, and Rehmanniae Radix Siccus is effective against thirst occurring after fever disease, and a disease symptomized by thirst due to the heat of the bowels, and acts to stop hemoptysis and nasal bleeding.

As used herein, the term “drying” is performed through hot air dry (AD), cold air dry, vacuum dry (VD), spray dry (SD), and freeze dry (FD) methods, and in the present disclosure, drying was carried out by using a hot air dry method. Hot air drying is drying using hot air, wherein air with low humidity is heated to allow to flow between objects to be dried, thereby evaporating and drying the moisture contained in the objects to be dried.

In the present disclosure, Rehmannia may be Rehmanniae Radix, or the roots may be selected for use, but is not limited thereto.

As used herein, the term “Poria” corresponds to fungus of Poria cocos Wolf, which are fungus belonging to the family Polyporaceae, and is parasitic on the root of trees, such as pine trees, in soil. It has a sclerotium size of 10-30 cm and is round-shaped, longish or mass-shaped. The surface is reddish-brown, light brown or dark brown in color and is generally rough, and in some cases, the root bark is broken. The fresh is white and gradually turns rose pink. The white one is called white Poria, and the red one is red Poria. Also, a portion of Poria cocos that the pine tree root penetrates is called Hoelen cum Pini Radix. These are medicinal herbs, and have tonic, diuretic, and sedative effects and thus are used for renal diseases, cystitis, and urethritis.

As used herein, the term “Hawthorn fruit” corresponds to a medicinal herb obtained by drying ripen fruits of Crataegus pinnatifida Bge, Crataegus cuneata Sieb. et Zucc, Crataegus pinnatifida var. major N. E. Br, or Crataegus pinnatifida for. psilosa (C. K. Schneid.) Kitag, or closely related congeneric plant, which is a perennial herb belonging to the family Rosaceae. Hawthorn fruit is often called San-sa since the fruit tastes like apples and is red in color and thus is like a small apple. Hawthorn fruit was also called Jujubae Fructus since the shape thereof is similar to that of red jujube. Hawthorn fruit was reported to have cardiotonic, blood circulation improving, and blood pressure lowering actions.

Hawthorn fruit may be used by fruits thereof, but is not limited thereto.

As used herein, the term “Platycodon” corresponds to a perennial herb (Platycodon grandiflorum (Jacq.) A. DC.) belonging to the family Campanulaceae, about 40-100 cm in height. The leaves are alternate and oval in shape. The root is chubby, and the stalks emerge individually or in groups. White or sky-blue flowers bloom in July or August, and the fruit is a capsule type. The root is called “Platycodon root” and is edible or used as a medicinal herb for expectorant or antitussive purposes.

Platycodon may be used by roots thereof, but is not limited thereto.

As used herein, the term “Astragalus” is a perennial plant belonging to the family Leguminosae of the order Rosales of the dicotyledonous plant, and originates from the main peel of the root, peeled and dried. The root is thin and long columnar, 30-100 cm in length, and 7-20 mm in diameter. The small lateral roots are sparsely attached thereto without being branched, and are slightly twisted near the root top portion and the residue of the stem remains. The outer surface of the root is light grayish yellow—light yellowish brown in color, and the grayish brown cork layer remains in places, and the regular rough vertical folds and horizontal cortex-like appearance are shown. The texture is dense and hard to break, and the broken side is fibrous. Astragalus is widely distributed in Asian regions, such as Korea and China, and in Europe, such as Russia and Bulgaria, and is an important medicinal herb that is frequently used in the 300 types of prescriptions including Hwanggigeonjungtang, Hwanggigyejiomultang, Sipjeondaebotang, Banggihwanggitang, and Bojungikgitang. Astragalus has isoflavonoids and triterpene saponins as main ingredients and contains cannabanins, phenolic glycosides, and the like, and thus has been reported to have effects, such as diuretic action, a blood pressure lowering action, tonic action to preserve vigor, cellular and humoral immunity enhancing action, anti-inflammatory action, and reduction of alkaline RNase activity in the liver and spleen.

Astragalus may be used by roots thereof, but is not limited thereto.

As used herein, the term “extraction” is a method in which useful soluble components contained in liquid or solid raw materials are separated by dissolution in a solvent, and examples thereof include room-temperature extraction, cold extraction, hot water extraction, ultrasonic extraction, steam extraction, reflux cooling extraction, and extraction under reduced or increased pressure. In an embodiment of the present disclosure, reflux extraction was conducted. Reflux extraction is an extraction method in which a cooling device is attached to the top portion of an extraction container to cool and concentrate the evaporating solvent, thereby minimizing the amount of the evaporating solvent from the solution.

As used herein, the term “extract” includes a crude solvent extract, a specific solvent soluble extract (solvent fraction), and a solvent fraction of a crude solvent extract, and the extract may be in a solution, concentrate, or powder state.

The extract may be a crude extract obtained by extraction with at least one solvent selected from the group consisting of water and linear or branched alcohols having 1 to 4 carbon atoms, wherein the alcohols may be at least one selected from the group consisting of methanol, ethanol, propanol, and butanol, but is not limited thereto.

The extract may be a solvent fraction obtained by fractionating the solvent crude extract with an additional solvent, and for example, may be a solvent fraction obtained by fractionating the solvent crude extract with at least one selected from the group consisting of formic acid, acetonitrile, ethyl ether, ethyl acetate, and butanol, but is not limited thereto.

As used herein, the term “concentration” refers to an operation in which the concentration of solids is increased by removing a liquid from a liquid material with a high liquid content. In an embodiment of the present disclosure, concentration was conducted by using concentration under reduced pressure. Concentration under reduced pressure is a method in which the concentration of a solute is increased by lowering the boiling point of a solvent under reduced pressure to quickly evaporate the solvent.

As used herein, the term “virus” means a toxic substance in Latin, and refers to a group of infectious pathogenic particles that pass through a bacteriological filter bed (0.22 um). The virus used in an embodiment of the present disclosure is influenza virus (PR8-GFP).

As used herein, the term “macrophages” means main cells responsible for innate immunity, and refers to a type of phagocytic leukocytes that absorbs and digests cellular tissues or non-protein, such as foreign substances, microorganisms, and cancer cells, present in a healthy body. The macrophages used in an embodiment of the present disclosure are RAW 264.7 cells.

In the present disclosure, the pharmaceutical composition can exhibit antiviral activity against at least one selected from the group consisting of influenza virus, corona virus, vesicular stomatitis virus, and Newcastle disease virus, and activity to alleviate, prevent, or treat a viral infection caused by at least one selected from the group.

The pharmaceutical composition may contain the complex extract at a concentration of 0.01-20.00 g, 0.01-15.00 g, 0.01-10.00 g, 0.01-5.00 g, 0.01-2.00 g, 0.10-20.00 g, 0.10-15.00 g, 0.10-10.00 g, 0.10-5.00 g, 0.10-2.00 g, 0.50-20.00 g, 0.50-15.00 g, 0.50-10.00 g, 0.50-5.00 g, 0.50-2.00 g, 1.00-20.00 g, 1.00-15.00 g, 1.00-10.00 g, 1.00-5.00 g, or 1.00-2.00 g as an adult daily intake, but is not limited thereto.

In the present disclosure, the enhancement of innate immunity may be induced by the generation of immune cytokines, such as tumor necrosis factor (TNF)-α and interleukin (IL)-6.

As used herein, the term “TNF-α” refers to a cytokine secreted in various immune cells in vivo and is a factor that plays an important role in various immune-mediated inflammatory diseases. TNF-α mediates various types of cellular actions, such as proliferation, survival, differentiation, and apoptosis (cell death), and plays an important role in the induction and maintenance of inflammatory immune responses.

As used herein, the term “IL-6” refers to a pro-inflammatory cytokine that plays a key role in the immune-mediated inflammatory diseases, while signaling for inflammatory responses through the JAK/STAT pathway involved in many chronic inflammatory diseases. IL-6 is a factor that is needed for differentiating naïve T cells into Th17 cells.

The pharmaceutical composition of the present disclosure may be used as a pharmaceutical composition containing a pharmaceutically effective amount of the complex extract and/or a pharmaceutically acceptable carrier.

As used herein, the term “pharmaceutically effective amount” refers to an amount that is sufficient to attain the effect or activity of the aforementioned complex extract.

The pharmaceutically acceptable carrier contained in the pharmaceutical composition of the present disclosure is conventionally used for the formulation, and examples thereof may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, but are not limited thereto. The pharmaceutical composition of the present disclosure may further contain, in addition to the above ingredients, a lubricant, a wetting agent, a sweetening agent, a flavoring agent, an emulsifier, a suspending agent, a preservative, and the like.

The pharmaceutical composition according to the present disclosure may be administered to mammals including humans through various routes. The manner of administration may be any manner that is conventionally used, and the administration may be conducted through an oral, dermal, intravenous, intramuscular, or subcutaneous route, and for example, the administration may be conducted through an oral route.

The appropriate dose of the pharmaceutical composition of the present disclosure varies depending on factors, such as a formulating method, a manner of administration, patient's age, body weight, sex, and morbidity, food, a time of administration, a route of administration, an excretion rate, and response sensitivity. An ordinarily skilled practitioner can easily determine and prescribe the dose that is effective for the desired treatment or prevention.

The pharmaceutical composition of the disclosure may be formulated into a unit dosage form or may be prepared in a multi-dose container by using a pharmaceutically acceptable carrier and/or excipient according to a method that can be easily implemented by a person having an ordinary skill in the art to which the present disclosure belongs. The formulation may be in the form of a solution in an oily or aqueous medium, a suspension, an emulsion, an extract, a powder, granules, a tablet, a capsule, or a gel (e.g., a hydrogel), and may further contain a dispersant or a stabilizer.

In accordance with still another aspect of the present disclosure, there is provided a health functional food for enhancement of innate immunity, antiviral use, or prevention or treatment of a viral infection, the health functional food containing a second complex extract of two or more selected from the group consisting of Codonopsis, Rehmannia, Ginseng, Platycodon, Poria, Hawthorn fruit, and Astragalus.

The second complex extract may contain 1.25-5.0 wt % of Codonopsis, 12.50-50.0 wt % of Rehmannia, 1.25-5.00 wt % of Ginseng, 6.85-27.00 wt % of Platycodon, 2.50-10.00 wt % of Poria, 0.65-2.50 wt % of Hawthorn fruit, and 25.0-80.0 wt % of Astragalus, and preferably, 2.00-3.00 wt % of Codonopsis, 20.00-30.00 wt % of Rehmannia, 2.00-3.00 wt % of Ginseng, 10.00-20.00 wt % of Platycodon, 3.00-8.00 wt % of Poria, 1.00-1.50 wt % of Hawthorn fruit, and 40.0-60.0 wt % of Astragalus, but is not limited thereto.

The second complex extract may contain at least one compound selected from the group consisting of chlorogenic acid, ginsenoside Rg1, calycosin, ginsenoside Rb1, ginsenoside Rd, astragaloside II, astragaloside I, and polygalacin D, and for example, chlorogenic acid or ginsenoside Rd, but is not limited thereto.

In the present disclosure, the health functional food can exhibit antiviral activity against at least one selected from the group consisting of influenza virus, corona virus, vesicular stomatitis virus, and Newcastle disease virus, and activity to alleviate a viral infection caused by at least one selected from the group.

When the food composition of the present disclosure is used as a health functional food additive, the food composition may be added as it is, or may be used along with other food or food ingredients, and may be appropriately used by a conventional method. Typically, the food composition of the present disclosure may be added at an amount of 10-60 wt %, preferably 30-50 wt %, relative to the health functional food in the manufacture of food or beverage, but is not limited thereto.

The type of food is not particularly limited. Examples of the food to which the material may be added include meat, sausage, bread, chocolate, candies, snacks, confectioneries, pizza, instant noodles, other noodles, gums, dairy products including ice creams, various soups, soft drinks, tea, drinks, alcoholic drinks, vitamin complexes, and the like, and include all foods in a typical sense.

The beverage may contain as additive ingredients various kinds of flavoring agents or natural carbohydrates. The aforementioned natural carbohydrates may include monosaccharides, such as glucose and fructose, disaccharides, such as maltose and sucrose, natural sweeteners, such as dextrin and cyclodextrin, and synthetic sweeteners, such as saccharin and aspartame. The proportion of the natural carbohydrates may be appropriately determined by the selection of a person skilled in the art.

In addition to the aforementioned ingredients, the food composition of the present disclosure may contain various types of nutrient supplements, vitamins, electrolytes, flavors, colorants, pectic acid and salts thereof, alginic acid and salts thereof, organic acids, protective colloid thickeners, pH adjusters, stabilizers, preservatives, glycerin, alcohols, carbonating agents used in carbonated drinks, and the like Furthermore, the food composition of the present disclosure may contain fruit flesh for manufacturing natural fruit juices, fruit juice drinks, and vegetable drinks. These ingredients may be used either alone or in combination. The proportions of these ingredients may also be appropriately selected by a person skilled in the art.

In accordance with still another aspect of the present disclosure, there is provided a method for enhancement of innate immunity, antiviral use, or alleviation, prevention, or treatment of a viral infection, the method including:

administering to a subject a composition containing a first complex extract of Codonopsis and Rehmannia.

In accordance with still another aspect of the present disclosure, there is provided a method for enhancement of innate immunity, antiviral use, or alleviation, prevention, or treatment of a viral infection, the method including:

administering to a subject a composition containing a second complex extract of two or more selected from the group consisting of Codonopsis, Rehmannia, Ginseng, Platycodon, Poria, Hawthorn fruit, and Astragalus.

The natural complex extracts of the present disclosure exhibit effects of preventing and treating an infection with a virus, such as influenza virus or corona virus by enhancing innate immunity and promoting the secretion of interferon induced by inflammatory factors (cytokines), and thus can be significantly helpful in the effective enhancement of immunity or the prevention, treatment, and alleviation of viral infections, and are greatly characterized by having few side effects.

### BRIEF DESCRIPTION OF THE DRAWINGS

FIG. 1A illustrates images showing the rates of influenza virus (PR8) infection on macrophages treated with a complex extract according to an exemplary embodiment of the present disclosure.

FIG. 1B illustrates a graph showing the rates of influenza virus infection on macrophages treated with a complex extract according to an exemplary embodiment of the present disclosure.

FIG. 2A illustrates a graph showing the amounts of production of tumor necrosis factor (TNF)-α in macrophages treated with a complex extract according to an exemplary embodiment of the present disclosure.

FIG. 2B illustrates a graph showing the amounts of production of interleukin (IL)-6 in macrophages treated with a complex extract according to an exemplary embodiment of the present disclosure.

FIG. 3 illustrates a graph comparing the contents of solids of complex extracts depending on the extraction temperature and extraction time according to an exemplary embodiment of the present disclosure.

FIG. 4A illustrates a graph showing effective cytotoxic concentration of OCD20015-V009 in RAW 264.7 and MDCK cells.

FIG. 4B illustrates images confirming the influenza A virus (IAV) replication inhibitory effects of OCD20015-V009 in RAW 264.7 cells according to an exemplary embodiment of the present disclosure.

FIG. 4C illustrates a graph confirming, through flow cytometry, the IAV replication inhibitory effects of OCD20015-V009 in RAW 264.7 cells according to an exemplary embodiment of the present disclosure.

FIG. 4D illustrates a graph confirming the plaque formation reducing effects of OCD2015-V009 in IAV-infected MDCK cells according to an exemplary embodiment of the present disclosure.

FIG. 4E illustrates images confirming the protein producing efficiency of OCD20015-V009 in IAV-infected RAW 264.7 cells according to an exemplary embodiment of the present disclosure.

FIG. 4F illustrates a graph confirming the protein producing efficiency of OCD20015-V009 in IAV-infected RAW 264.7 cells according to an exemplary embodiment of the present disclosure.

FIG. 5A illustrates a graph showing the daily percentage of survival up to 10 days post-infection depending on OCD20015-V009 pre-treatment in IAV-infected mice according to an exemplary embodiment of the present disclosure.

FIG. 5B illustrates images showing the histopathological results in the lung tissue depending on OCD20015-V009 pre-treatment in IAV-infected mice according to an exemplary embodiment of the present disclosure.

FIG. 6A illustrates a graph showing the inflammatory cytokine TNF-α inducing effects by OCD20015-V009 treatment in RAW 264.7 cells according to an exemplary embodiment of the present disclosure.

FIG. 6B illustrates a graph showing the inflammatory cytokine IL-6 inducing effect by OCD20015-V009 treatment in RAW 264.7 cells according to an exemplary embodiment of the present disclosure.

FIG. 6C illustrates a graph showing the inflammatory cytokine TNF-α inducing effect at 24 h after OCD20015-V009 treatment in RAW 264.7 cells according to an exemplary embodiment of the present disclosure.

FIG. 6D illustrates a graph showing the inflammatory cytokine IL-6 inducing effect at 24 h after OCD20015-V009 treatment in RAW 264.7 cells according to an exemplary embodiment of the present disclosure.

FIG. 6E illustrates Western blot images showing the activation levels of type I IFN signaling molecules by OCD20015-V009 pre-treatment in RAW 264.7 cells according to an exemplary embodiment of the present disclosure.

FIG. 6F illustrates graphs showing the activation levels of type I IFN signaling molecules by OCD20015-V009 pre-treatment in RAW 264.7 cells according to an exemplary embodiment of the present disclosure.

FIG. 7A illustrates a graph showing the time-specific expression levels of ISG-15 gene by OCD20015-V009 pre-treatment in RAW 264.7 cells according to an exemplary embodiment of the present disclosure.

FIG. 7B illustrates a graph showing the time-specific expression levels of ISG-20 gene by OCD20015-V009 pre-treatment in RAW 264.7 cells according to an exemplary embodiment of the present disclosure.

FIG. 7C illustrates a graph showing the time-specific expression levels of IFN-β gene by OCD20015-V009 pre-treatment in RAW 264.7 cells according to an exemplary embodiment of the present disclosure.

FIG. 7D illustrates a graph showing the time-specific expression levels of ISG-56 gene by OCD20015-V009 pre-treatment in RAW 264.7 cells according to an exemplary embodiment of the present disclosure.

FIG. 7E illustrates a graph showing the time-specific expression levels of TNF-α gene by OCD20015-V009 pre-treatment in RAW 264.7 cells according to an exemplary embodiment of the present disclosure.

FIG. 8A illustrates graphs showing substances identified by UV chromatograms and total ion chromatograms of OCD20015-V009 according to an exemplary embodiment of the present disclosure.

FIG. 8B illustrates graphs showing substances identified by PRM chromatograms of OCD20015-V009 according to an exemplary embodiment of the present disclosure.

FIG. 9A illustrates images confirming the IAV replication inhibitory effects of eight compounds derived from OCD20015-V009 in RAW 264.7 cells according to an exemplary embodiment of the present disclosure.

FIG. 9B illustrates a graph confirming the IAV replication inhibitory effects of eight compounds derived from OCD20015-V009 in RAW 264.7 cells according to an exemplary embodiment of the present disclosure.

FIG. 9C illustrates the chemical formulas showing two main components identified in OCD20015-V009 separated according to an exemplary embodiment of the present disclosure.

FIG. 9D illustrates graphs confirming the protein producing efficiency of chlorogenic acid (CGA) and ginsenoside Rd in IAV-infected RAW 264.7 cells according to an exemplary embodiment of the present disclosure.

### DETAILED DESCRIPTION OF THE EXEMPLARY EMBODIMENTS

Hereinafter, the present disclosure will be described in more detail by the following examples. However, these examples are used only for illustration, and the scope of the present disclosure is not limited by these examples.

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

As described above, conventional immunotherapy agents are drugs that suppress the immune response, and the overdose thereof may cause symptoms, such as infectious diseases, allergic responses, itchiness, injection site erythema (including bleeding, bruising, erythema, pain, and swelling), breathing difficulties, and fever, and has a risk of exposure to viral infections due to the decline in immunity or the like resulting from immunoregulatory failure.

According to the present disclosure, solutions to the above-described problems have been sought by providing a composition for enhancement of innate immunity, antiviral use, or prevention or treatment of a viral infection, the composition containing as an active ingredient a complex extract of two or more selected from the group consisting of Codonopsis, Rehmannia, Ginseng, Platycodon, Poria, Hawthorn fruit, and Astragalus. Such a composition was confirmed to have excellent effects on immunity and viral infection alleviation by promoting the interferon secretion in the innate defense immune system and thus can be greatly helpful in the enhancement of innate immunity, antiviral use, or the prevention, treatment, or alleviation of a viral infection.

### Preparative Example 1: Preparation of First Complex Extract 1-1. Preparation of Example 1

To prepare a first complex extract, raw materials were prepared by 7.4 wt % of Codonopsis and 92.6 wt % Rehmannia, which were previously cut to appropriate sizes, and primary distilled water having 10 times the total weight of the raw materials was added thereto, followed by reflux extraction at 100° C. for 120 minutes. The extracted solution was primarily filtered through a 0.45-um filter bed and secondarily filtered through a 0.22 um-filter bed to remove precipitates, concentrated under reduced pressure at 45-55° C., and then freeze-dried at −80° C. and 5 mTorr to be powdered. The powdered first complex extract was dispensed at 1 g in each 1.5 ml Ep-tube and stored at −20° C., and this was designated as Example 1 and used for testing.

### 1-2. Preparation of Example 2

The preparation was carried out in the same manner as in Example 1, except that the raw materials were prepared by 6.8 wt % of Codonopsis, 8.0 wt % of Ginseng, and 85.2 wt % of Rehmannia and the powdered complex extract was designated as Example 2 and used for testing.

### 1-3. Preparation of Example 3

The preparation was carried out in the same manner as in Example 1, except that the raw materials were prepared by 6.2 wt % of Codonopsis, 77.5 wt % of Rehmannia, and 16.3 wt % of Platycodon and the powdered complex extract was designated as Example 3 and used for testing.

### 1-4. Preparation of Example 4

The preparation was carried out in the same manner as in Example 1, except that the raw materials were prepared by 5.0 wt % of Codonopsis, 5.9 wt % of Ginseng, 62.7 wt % of Rehmannia, 12.1 wt % of Poria, 1.1 wt % of Hawthorn fruit, and 13.2 wt % of Platycodon and the powdered complex extract was designated as Example 4 and used for testing.

### Preparative Example 2: Preparation of OCD20015-V009

OCD20015-V009 was prepared by immersing 2.50 wt % of Codonopsis, 25.00 wt % of Rehmannia, 2.50 wt % of Ginseng, 13.75 wt % of Platycodon, 5.00 wt % of Poria, 1.25 wt % of Hawthorn fruit, and 50.00 wt % of Astragalus (total of 2000 g), all of which were dried, in 10 L of distilled water, followed by hot-water extraction at 115° C. for 3 hours. The resultant extract was filtered through a 150-um sieve and then freeze-dried. The yield of the second complex extract was 14.1% (283.7 g). The extract was stored in desiccators at 4° C. until further use in KM-Application Center herbarium (registration number, #OCD2020-1) of Korea Institute of Oriental Medicine (KIOM).

### Test Example 1: Analysis of Antiviral Activity of First Complex Extracts in Mouse Macrophage Line

The antiviral activity of the first complex extracts on the green fluorescent protein (GFP)-labeled influenza virus (PR8-GFP) was analyzed.

Specifically, RAW 264.7 cells (macrophages) were seeded at 2×10⁵ cells/well in 24-well tissue culture (TC) plates and incubated in RPMI medium supplemented with 1% fetal bovine serum (FBS) for 24 hours. Thereafter, the cells were treated with a sample of each of Examples 1 to 4 prepared in Preparative Example 1 at 200 μg/ml for 18 hours.

After the sample treatment for 18 hours, the cells were infected with an inoculum containing the influenza virus (A/PR/8/34-GFP) at a multiplicity of infection (MOI) of 1.0 for 2 hours. After 2 hours of infection, the inoculum was removed and the cells were washed three times with phosphate-buffered saline (PBS). The cells were again incubated in RPMI medium for 24 hours, and then the degrees of infection with the inoculated virus were investigated. Thereafter, a fluorescence microscope and flow cytometry capable of confirming GFP expression were used to investigate the inhibition of influenza virus proliferation.

RPMI medium supplemented with 1% FBS was used as a negative control (vehicle), and a virus-infected medium treated with mouse IFN-β (1,000 units/ml) was used as a positive control (IFN). The results are shown in Table 1.

TABLE 1

| | | | Example | Example | Example | Example | |
|---|---|---|---|---|---|---|---|
| | CON | Vehicle | 1 | 2 | 3 | 4 | IFN-β |
| PR8- | − | + | + | + | + | + | + |
| GFP | | | | | | | |
| GFP | 0.97 | 12.74 | 4.54 | 3.39 | 3.64 | 2.10 | 1.45 |

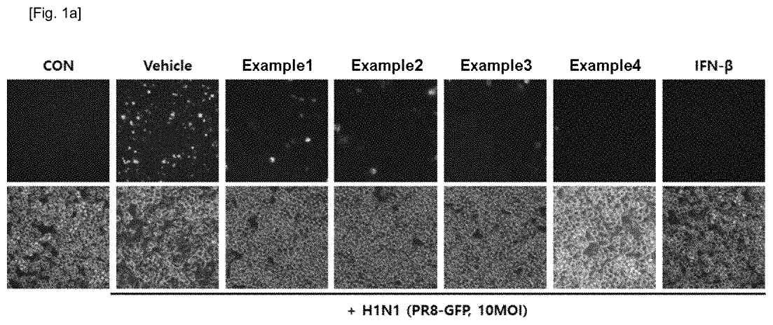

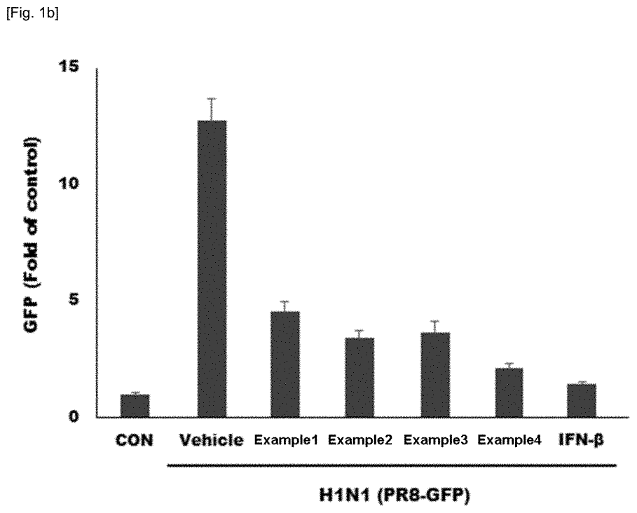

As can be confirmed in Table 1 and FIGS. 1A and 1B, as a result of infecting the cells treated with the samples of Examples 1 to 4 with the influenza virus, the influenza virus infection rates were remarkably dropped, and especially, the complex extract of Example 4 (OCD20015) showed no significant difference compared with the cells treated with the positive control IFN-β. These results indicate that the complex extracts increase the resistance to the virus by enhancing the immunity of cells.

### Test Example 2: Analysis of Innate Immunity Enhancing Activity of First Complex Extracts in Mouse Macrophage Line

To investigate the innate immunity enhancing activity of the first complex extracts, immune cytokines were measured.

Specifically, RAW 264.7 cells were incubated at 2×10⁵ cells/well in 96-well TC plates, and then were treated with each of the complex extracts prepared in Examples 1 to 4 at 200 μg/ml in RPMI medium supplemented with 1% FBS. After 24 hours of the extract treatment, the supernatant was collected to measure tumor necrosis factor (TNF)-α and interleukin (IL)-6 by using sandwich ELISA. The cells treated with 200 ng/ml lipopolysaccharide (LPS) were used as a positive control.

After 24 hours of the treatment with the complex extracts of Examples 1 to 4, the immune cytokines were measured, and the results are shown in Table 2.

TABLE 2

| | | | Example | Example | Example | Example |
|---|---|---|---|---|---|---|
| | CON | LPS | 1 | 2 | 3 | 4 |
| TNF-α (pg/ml) | 0 | 16,055 | 10,505 | 12,333 | 11,524 | 13,021 |
| IL-6 (pg/ml) | 0 | 26,002 | 12,511 | 13,584 | 15,511 | 16,003 |

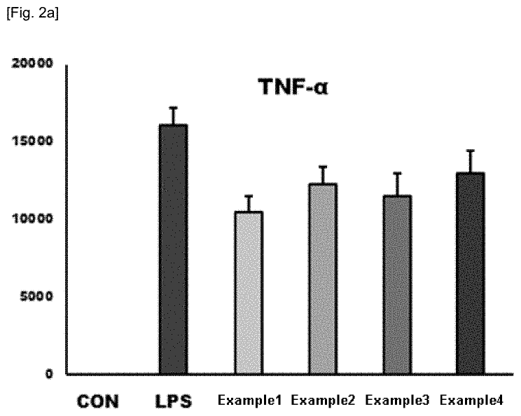

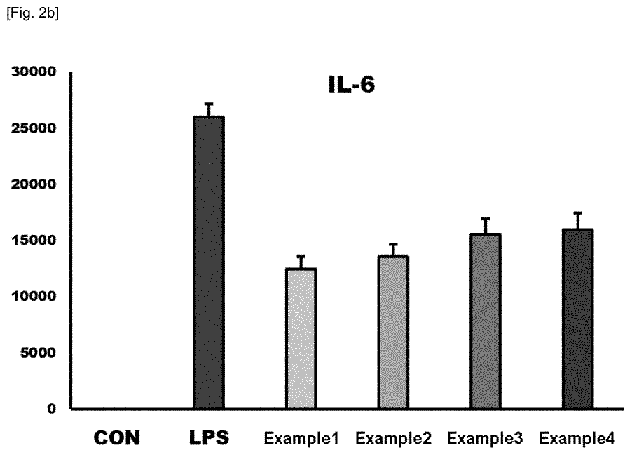

As can be confirmed in Table 2 and FIGS. 2A and 2B, as a result of treating with the complex extracts of Examples 1 to 4 for 24 hours, the amounts of production of the immune cytokines were remarkably increased compared with the control. Especially, the complex extract (OCD20015) of Example 4 showed a high-degree increase compared with the other extracts. These results indicate that the OCD20015 complex extract has an effect on the enhancement of immunity of cells.

### Preparative Example 3: Preparation of Examples 5 to 49

To investigate the solid contents at the time of extraction, first complex extracts were prepared under different conditions. The raw materials were prepared at the same ratio as in “Preparative Example 1-4” for preparing Example 4, and primary distilled water having 10 times the total weight of the raw materials was added thereto, followed by hot water extraction at 20, 30, 40, 50, 60, 70, 80, 90, and 100° C. for 1, 2, 4, 8, 12, 24, 48, 72, 96, and 120 hours. The extraction solutions were filtered through 1-um filter beds. The extraction conditions for each case are shown in Table 3 below.

TABLE 3

| | Temperature (° C.) | Time (H) |
|---|---|---|
| Example 5 | 40 | 1 |
| Example 6 | 40 | 2 |
| Example 7 | 40 | 4 |
| Example 8 | 40 | 12 |
| Example 9 | 40 | 24 |
| Example 10 | 40 | 48 |
| Example 11 | 40 | 72 |
| Example 12 | 40 | 96 |
| Example 13 | 40 | 120 |
| Example 14 | 60 | 1 |
| Example 15 | 60 | 2 |
| Example 16 | 60 | 4 |
| Example 17 | 60 | 12 |
| Example 18 | 60 | 24 |
| Example 19 | 60 | 48 |
| Example 20 | 60 | 72 |
| Example 21 | 60 | 96 |
| Example 22 | 60 | 120 |
| Example 23 | 80 | 1 |
| Example 24 | 80 | 2 |
| Example 25 | 80 | 4 |
| Example 26 | 80 | 12 |
| Example 27 | 80 | 24 |
| Example 28 | 80 | 48 |
| Example 29 | 80 | 72 |
| Example 30 | 80 | 96 |
| Example 31 | 80 | 120 |
| Example 32 | 100 | 1 |
| Example 33 | 100 | 2 |
| Example 34 | 100 | 4 |
| Example 35 | 100 | 12 |
| Example 36 | 100 | 24 |
| Example 37 | 100 | 48 |
| Example 38 | 100 | 72 |
| Example 39 | 100 | 96 |
| Example 40 | 100 | 120 |
| Example 41 | 120 | 1 |
| Example 42 | 120 | 2 |
| Example 43 | 120 | 4 |
| Example 44 | 120 | 12 |
| Example 45 | 120 | 24 |
| Example 46 | 120 | 48 |
| Example 47 | 120 | 72 |
| Example 48 | 120 | 96 |
| Example 49 | 120 | 120 |

### Test Example 3: Setting of Extraction Conditions Through Comparison of Solid Content

The extracts of Examples 5 to 49 obtained by extraction according to Preparative Example 3 were subjected to solid content measurement and comparison. From each of the extracts, 3-4 g of a sample was taken, and then measured for solid content (%, g/g) at 105° C. by an infrared moisture analyzer model FD-720, and the results are shown in Table 4 below.

TABLE 4

| | Solid content (%) |
|---|---|
| Example 5 | 3.6 |
| Example 6 | 3.98 |
| Example 7 | 4.21 |
| Example 8 | 4.23 |
| Example 9 | 4.25 |
| Example 10 | 4.28 |
| Example 11 | 4.31 |
| Example 12 | 4.34 |
| Example 13 | 4.34 |
| Example 14 | 4.3 |
| Example 15 | 4.69 |
| Example 16 | 4.99 |
| Example 17 | 5.01 |
| Example 18 | 5.05 |
| Example 19 | 5.09 |
| Example 20 | 5.15 |
| Example 21 | 5.19 |
| Example 22 | 5.19 |
| Example 23 | 4.41 |
| Example 24 | 4.78 |
| Example 25 | 5.09 |
| Example 26 | 5.09 |
| Example 27 | 5.12 |
| Example 28 | 5.21 |
| Example 29 | 5.27 |
| Example 30 | 5.3 |
| Example 31 | 5.3 |
| Example 32 | 4.44 |
| Example 33 | 4.81 |
| Example 34 | 5.1 |
| Example 35 | 5.11 |
| Example 36 | 5.14 |
| Example 37 | 5.24 |
| Example 38 | 5.33 |
| Example 39 | 5.39 |
| Example 40 | 5.39 |
| Example 41 | 4.45 |
| Example 42 | 4.8 |
| Example 43 | 5.11 |
| Example 44 | 5.11 |
| Example 45 | 5.15 |
| Example 46 | 5.24 |
| Example 47 | 5.34 |
| Example 48 | 5.39 |
| Example 49 | 5.39 |

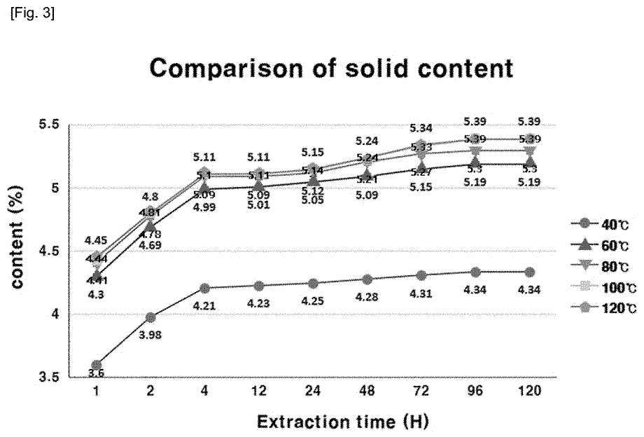

As can be confirmed in Table 4 and FIG. 3, the solid content to the extraction temperature was rapidly increased at 60° C. compared with 40° C., and the increase width was reduced at high temperatures, but the solid content was not changed at 100° C. or higher. The solid content to the extraction time was rapidly increased in a section of 1-4 hours, but the increase width was reduced after 4 hours, and the solid content was not changed after 96 hours.

Considering the solid content according to the extraction temperature and extraction time, the extraction conditions of at least 60° C. and at least four hours are needed. To further increase the solid content, the extraction temperature needs to be increased to 100° C. and the extraction time needs to be increased to 96 hours, but no solid content change was confirmed under higher temperature and more time conditions.

It is therefore considered that the extraction temperature and extraction time may be set within appropriate conditions in consideration of consumption costs in the mass production process stage.

### Test Example 4: Cytotoxicity of OCD20015-V009

RAW 264.7 (murine macrophage) and Madin-Darby canine kidney (MDCK, NBL-2) cells (American Type Culture Collection) were incubated in a medium (DMEM; Lonza, USA) containing 10% fetal bovine serum (FBS; Biotechnics Research, USA) and 1% penicillin and streptomycin (Cellgro, USA) at 37° C. in a 5% CO₂ incubator, and then used.

The cytotoxicity was determined using MTT assay. The RAW 264.7 and MDCK cells were seeded 1×10⁵ cells/well into 24-well plates, and OCD20015-V009 was added to each of the wells at a concentration of 0-400 μg/ml. MTT solutions were added to each well 24 h after the addition, and the cells were incubated for another 30 min. Subsequently, 1 mL of DMSO was added, and the absorbance at 540 nm was measured using an Epoch microplate reader (BioTek, USA).

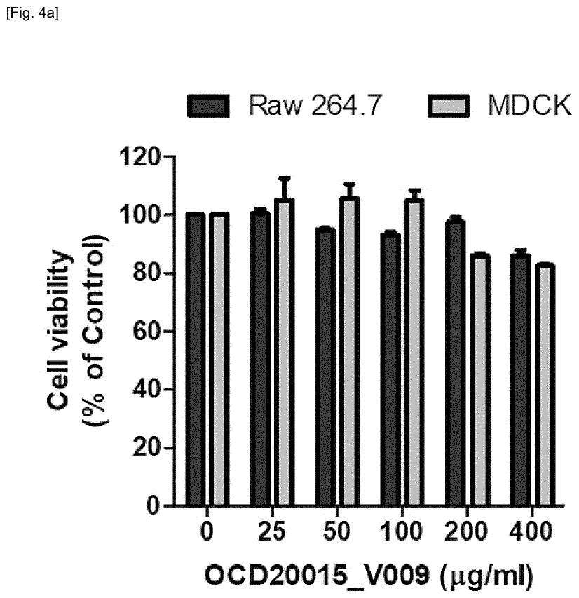

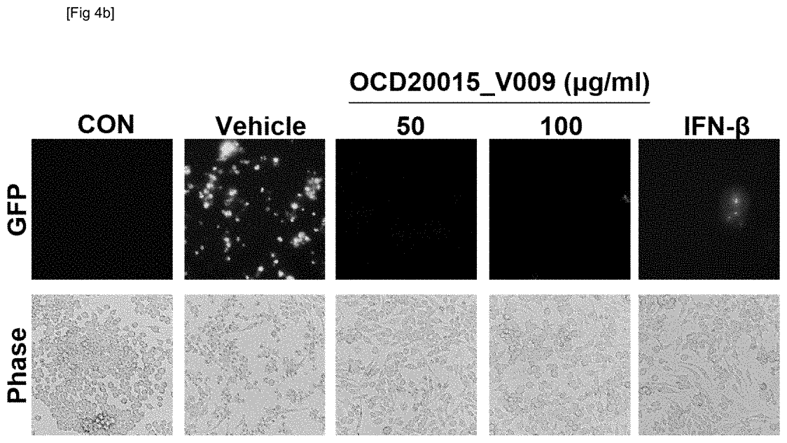

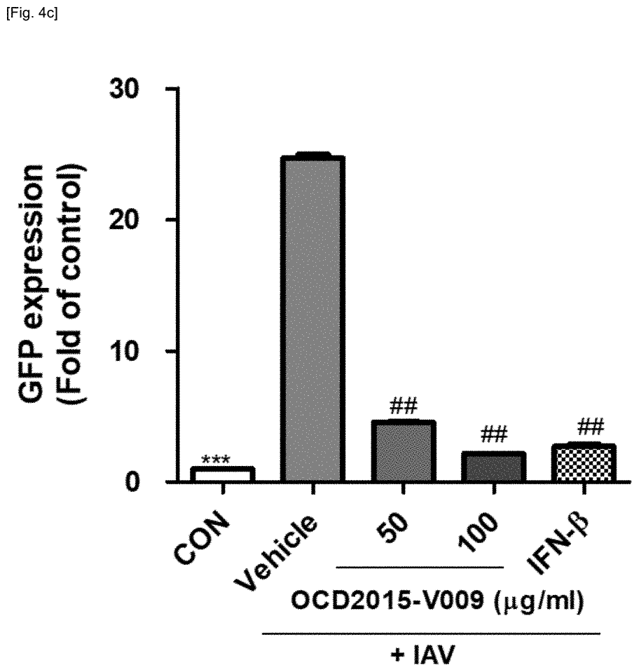

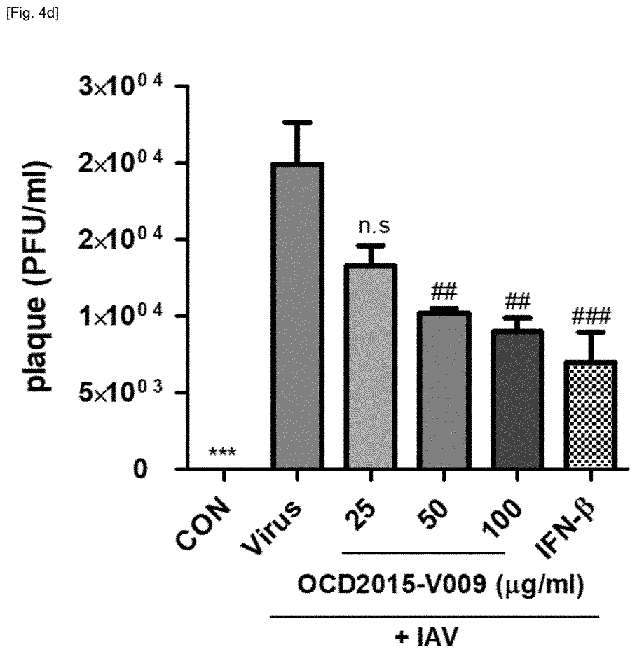

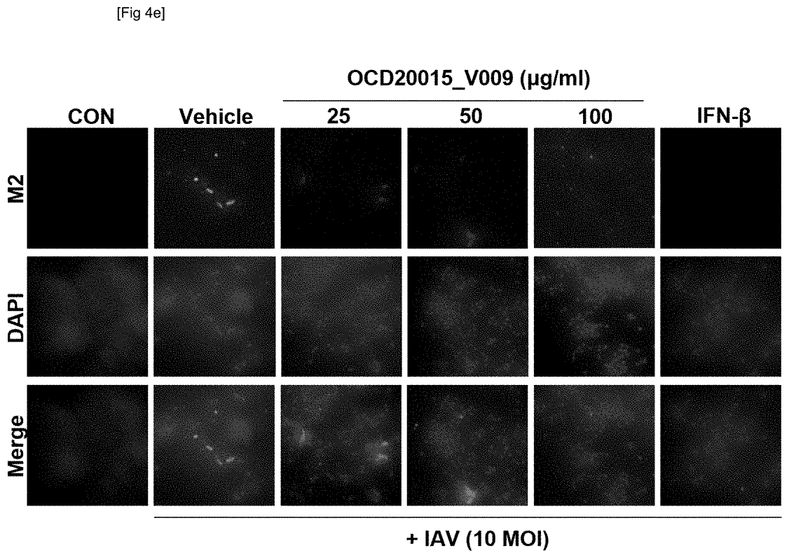

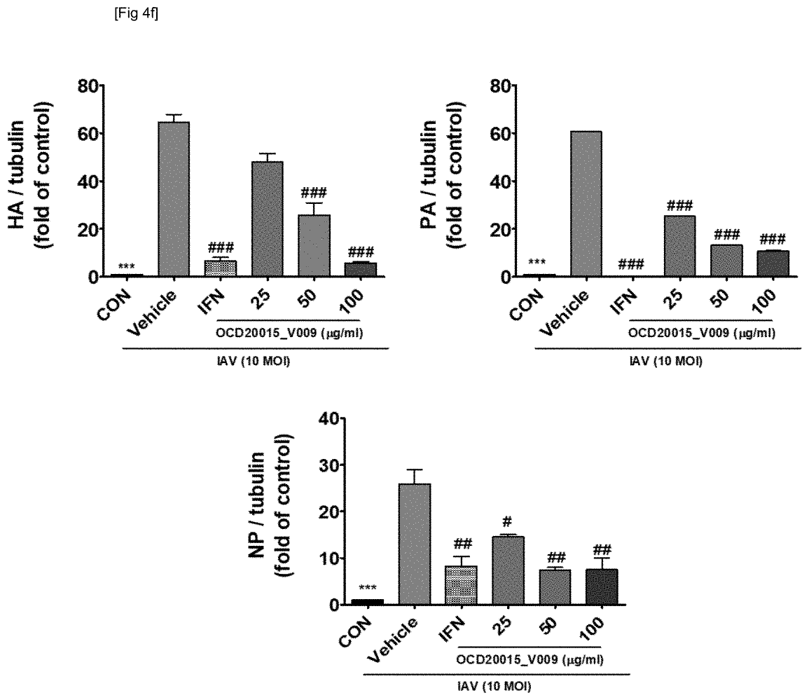

As can be confirmed in FIG. 4A, OCD20015-V009 showed no cytotoxicity in the RAW 264.7 cells. Therefore, subsequent tests were performed using OCD20015-V009 at less than 100 or 200 μg/ml.

### Test Example 5: IAV Infection Inhibitory Effect of OCD20015-V009

The viral replication inhibition of OCD20015-V009 was assayed using Influenza A (A/Puerto Rico/8/34; A/PR/8/34) from American Type Culture Collection (ATCC, VR-95™) and GFP-tagged A/PR/8/34 virus (A/PR/8/34-GFP). A/PR/8/34-GFP was briefly constructed by fusing the GFP gene to the C-terminal end of the nonstructural protein 1 open reading frame.

Specifically, RAW 264.7 cells were seeded 1×10⁵ cells/well into 24-well plates, and incubated for 18 hours in DMEM alone (for the untreated or virus-only group (Vehicle)), DMEM containing 1,000 U of recombinant mouse interferon (IFN-β (Sigma-Aldrich, USA), a positive control), or DMEM containing 50 and 100 μg/ml OCD20015-V009 in each well. The cells were then infected with A/PR/8/34-GFP at a multiplicity of infection (MOI) of 10. The GFP levels were measured 24-hour post-infection (hpi) at 200× magnification under a fluorescence microscope (Nikon, Japan). Thereafter, the cells were harvested using trypsinization, followed by fluorescence detection using flow cytometry (CytoFLEX, Beckman, USA).

The antiviral activity of OCD20015-V009 was examined by detecting GFP levels in RAW 264.7 cells after the suppression of A/PR/8/34-GFP replication.

As can be confirmed in FIG. 4B, the cells untreated with OCD20015-V009 had high GFP levels upon infection by A/PR/8/34-GFP. Conversely, the GFP level of RAW 264.7 cells pre-treated with OCD20015-V009 was considerably lower.

As can be confirmed in FIG. 4C, the replication of A/PR/8/34-GFP in RAW 264.7 cells was significantly decreased by 81.5 and 91.1% through OCD20015-V009 pre-treatment at 50 and 100 μg/ml, respectively, compared with the Vehicle.

For plaque analysis, Raw 264.7 cells were incubated in 24-well plates (1×10⁵ cells/ml) for 24 hours. Then, various concentrations of OCD2015-V009 were added and incubated at 37° C. for 18 hours. Following the reaction, the cells were infected with IAV, washed with phosphate-buffered saline (PBS), and then DMEM was added to the medium for 24 hours. Then, MDCK cells were infected for 2 hours with the Raw 264.7 cell culture supernatant containing viruses. Thereafter, MDCK monolayers were coated with 1.5% agarose in 2× complete DMEM and incubated with 5% CO₂ at 37° C. for 3 days. The cells were stained with a 1% crystal violet solution following incubation or infection, and plaques were counted.

As can be confirmed in FIG. 4D, that OCD20015-V009 reduced the plaque formation dose-dependently in MDCK cells.

Immunofluorescence (IF) staining was performed to validate the production of IAV proteins.

Specifically, RAW 264.7 cells seeded onto cover slides at 1×10⁵ cells/ml were incubated at 37° C. with 5% CO₂ for 24 hours. The RAW 264.7 cells were pre-treated with 25, 50, or 100 μg/ml OCD20015-V009, 1000 U/mL recombinant mouse interferon (IFN)-β, or only the medium (negative control) before viral adsorption. The cells were then incubated at 37° C. with 5% CO₂ for 18 hours, and then infected with A/PuertoRico/8/34 at the MOI of 10 for 2 hours.

After viral infection, the virus and medium were removed, and the cells were washed three times with phosphate-buffered saline (PBS). Then, a complete medium was added, and the cells were incubated at 37° C. with 5% CO₂. After 24 hours, the cells were washed with cold PBS, fixed with 4% paraformaldehyde at room temperature for 30 minutes, and permeabilized with 0.1% Triton-X100 in PBS for 15 minutes.

After blocking, the cells were incubated with a rabbit polyclonal antibody against M2 (1:250 in 3% BSA (Sigma-Aldrich); GeneTex, USA) at 4° C. overnight, washed three times with cold PBS, and incubated with an Alexa Fluor 568 goat anti-rabbit IgG antibody (1:500 in 3% BSA; Thermo Fisher) for 1 hour. The nuclei were visualized by staining with DAPI (0.5 μg/ml; Thermo Fisher) for 10 min. Then, the images were captured using a fluorescence microscope (Nikon).

The reduction of M2 in RAW 264.7 cells was observed by fluorescence microscopy using an M2-specific antibody. RAW 264.7 cells were stained with DAPI (blue), and the merged images represent M2 (red).

As can be confirmed in FIG. 4E, the cells pre-treated with OCD20015-V009 produced significantly less M2.

To validate the production of IAV proteins, the levels of influenza A virus proteins HA, PA, and NP in cell lysates were analyzed by Western blots.

Specifically, the RAW 264.7 cells seeded in 6-well plates at 1×10⁶ cells/well were incubated with OCD20015-V009 and LPS at 37° C. with 5% CO₂. Thereafter, the cells were harvested and lysed in RIPA buffer (Millipore, USA) containing protease and phosphatase inhibitors. The total protein content in the samples was normalized using Bradford's reagents. The proteins were separated using SDS-PAGE and transferred to a polyvinylidene fluoride membrane (Millipore). After blocking with 3% BSA, the blots were incubated with primary anti-STAT1, anti-TBK1, anti-phospho-STAT1, anti-phospho-TBK1 (Cell Signaling Technology, USA), anti-β-actin, M1, NA, NP, and PA antibodies (GeneTex, USA, 1:1,000 dilution) at 4° C. overnight. The level of tubulin was used as an internal control.

After the blots were washed three times in TBS-T, the blots were incubated with an HRP-conjugated secondary antibody (Cell Signaling Technology). The proteins were quantified using a ChemiDoc™ Touch Imaging System (Bio-Rad), and the relative intensities of protein bands were measured using ImageJ and quantified using the ImageJ software.

As can be confirmed in FIG. 4F, the production of HA, PA, and NP was significantly inhibited in RAW 264.7 cells pre-treated with 100 μg/ml OCD20015-V009 before infection with A/PR/8/34(H1N1)-GFP.

These data imply that OCD20015-V009 pre-treatment significantly inhibits IAV infection and viral protein production in RAW 264.7 cells. Therefore, OCD20015-V009 pre-treatment likely reduces influenza H1N1 viral protein production and inhibits infection.

### Test Example 6: IAV Infection Inhibition In Vivo by OCD20015-V009

The protective effects of OCD20015-V009 on IAV infection in BALB/c mice were investigated.

Five-week-old female BALB/c mice (Orient Bio Inc., Seongnam, South Korea) were acclimated for at least 1 week under standard housing conditions at the LAC of DGMIF and provided with a standard rodent chow diet and water ad libitum. For the oral inoculation of OCD20015-V009 and the IAV challenge, the mice were separated into four experimental groups of ten mice in each group and administered control, PBS, 100 or 300 mg/kg OCD20015-V009 with IAV, respectively. The group of mice without virus infection was used as a negative control.

The mice in each experimental group were orally administered PBS, 100 and 300 mg/kg OCD20015-V009 at a total volume of 200 μL once daily for 7 days before infection, respectively. The mice were infected intranasally with 20 μL of A/PR/8/34 in PBS at the 50% mouse lethal dose (LD50). The mice treated once daily with 100 or 300 mg/kg OCD20015-V009 maintained a relatively stable body weight with no significant clinical symptoms in this study.

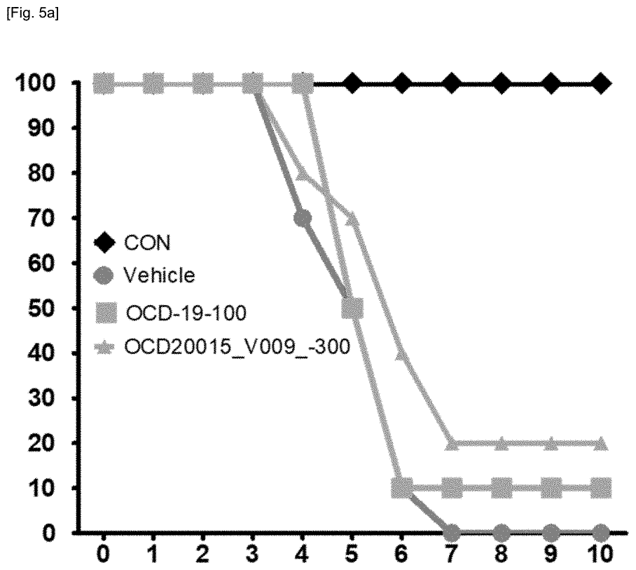

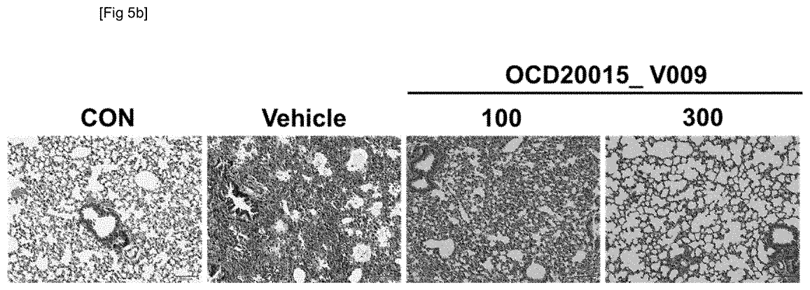

As can be confirmed in FIG. 5A, all untreated A/PR/8/34-infected mice were dead within 7 dpi. Contrarily, the mortality of the mice pre-treated with OCD20015-V009 after A/PR/8/34 infection was reduced.

Survival was monitored for 10 days (dpi) at fixed time points. At 7 dpi, three mice from each group were randomly selected and sacrificed to measure lung histopathology. Sampling was conducted at 7 dpi for staining to investigate histopathological changes caused by viral infection in the lung tissue samples cut from the untreated mice or the OCD20015-V009 pre-treated mice.

Specifically, the lung tissue samples were immediately fixed in paraffin-embedded neutral buffer containing 10% formalin and sliced to 4- to 6-μm sections using a microtome. The sections were mounted on a slide, stained with hematoxylin and eosin, and then examined under an optical microscope. The remaining mice were used to measure survival at 10 dpi.

As can be confirmed in FIG. 5B, lung inflammation or pathological changes were not found in the normal control group. However, bronchial epithelial cells were necrotized with thickened alveolar walls in the mice in the vehicle group. In addition, severe pulmonary congestion and lesions were observed. Also, the alveolar space was occupied with moderate inflammatory infiltrates of neutrophils, macrophages, and lymphocytes. However, lung samples from the mice pre-treated with 300 mg/kg OCD20015-V009 showed pulmonary congestion and lesion alleviation, indicating lower lung inflammation compared with the untreated mice.

### Test Example 7: Effects of OCD20015-V009 on Pro-Inflammatory Cytokine Production and Type I IFN Signaling Pathway Activation

Pro-inflammatory cytokines and type I IFN in pathway activation in RAW 264.7 murine macrophages are important in inducing immunoregulatory activities and antiviral responses. The immunoregulatory effects of herbal medicines for the treatment of IAV infection have been widely studied. Additionally, innate immune responses through the production of pro-inflammatory cytokines and type I IFN may be responsible for the antiviral action of OCD20015-V009. The effects of OCD20015-V009 on TNF-α and IL-6 secretion were evaluated using ELISA.

RAW 264.7 cells were seeded and incubated for 18 hours. The cells were treated with 200 ng/ml LPS or OCD20015-V009 at 50 or 100 μg/ml at 37° C. for 6 or 24 hours. The supernatant from each treatment group was harvested at 6 or 24 hours and centrifuged at 15,000 g for 10 minutes at 4° C. The clarified supernatants were dispensed into the enzyme-linked immunosorbent assay plates coated with the captured antibody of murine interleukin (IL)-6 or tumor necrosis factor (TNF)-α to measure cytokine secretion. The levels of the inflammatory cytokines TNF-α and IL-6 in the culture were measured using the ELISA antibody set (#88-7324-77 and #88-7064-77, eBioscience, USA).

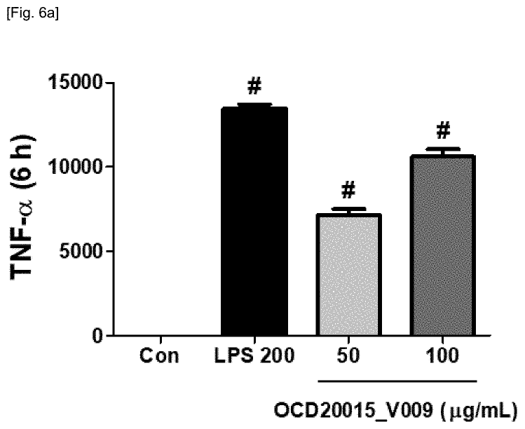

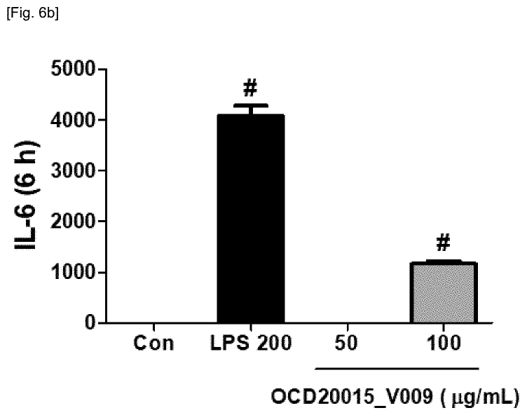

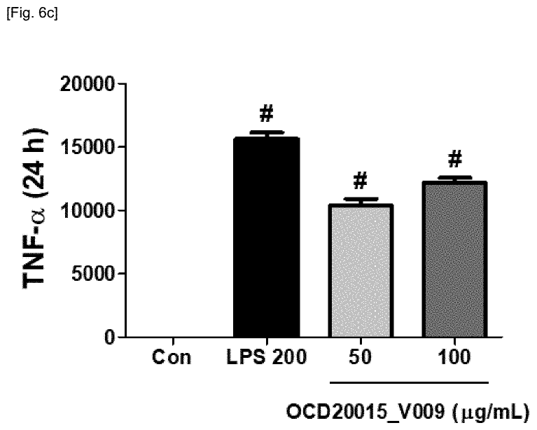

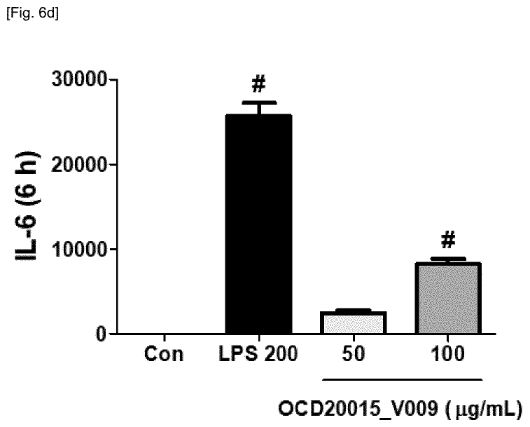

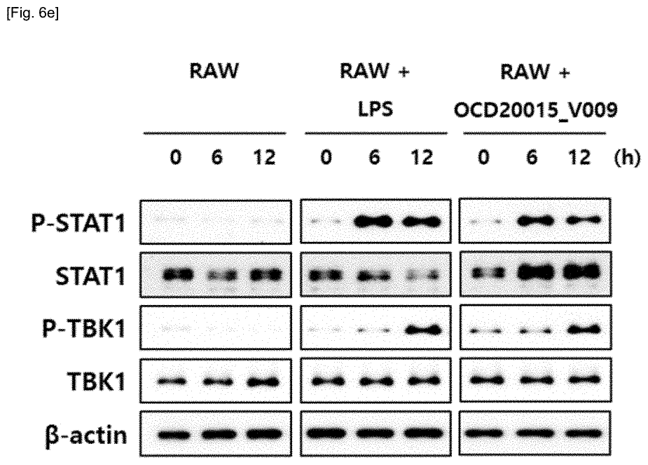

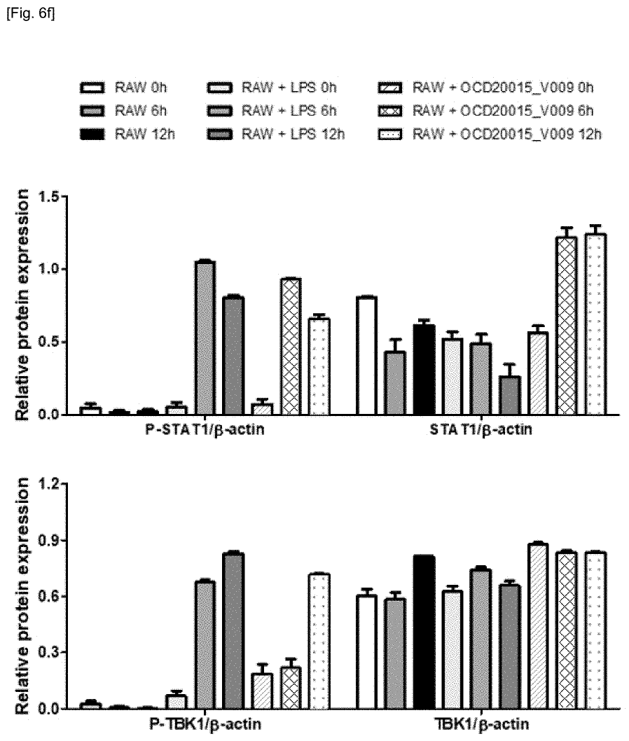

As can be confirmed in FIGS. 6A to 6D, the concentrations of secreted TNF-α and IL-6 increased by 10,597.4±768.9 and 1165.5±95.9 compared with the control in the treatment with 100 μg/ml OCD20015-V009 for 6 hours, respectively, and after 24 hours, 12,155.13±667 and 8,250.2±975.2 increased compared with control. These results indicate that OCD20015-V009 induces the antiviral response mediated by TNF-α and IL-6 in murine macrophages.

In addition, the Western blots were used to investigate the TBK1 and STAT1 phosphorylation in RAW 264.7 cells pre-treated with OCD20015-V009 to determine the effects of OCD20015-V009 on the activation of type I IFN signaling molecules. Western blotting of whole-cell lysates of macrophages treated with vehicle alone, 200 ng/ml LPS, or 200 μg/ml OCD20015-V009 was performed to assess the levels of the non-phosphorylated and phosphorylated forms of TANK-binding kinase 1 (TBK1), STAT1, and β-actin over time.

As can be confirmed in FIGS. 6E and 6F, the results show that the OCD20015-V009 treatment up-regulates the phosphorylation of STAT1 and TBK1 and these are key molecules in the type I IFN signaling pathway.

The interaction between OCD20015-V009 and IFN-stimulated genes in RAW 264.7 cells was further analyzed. The cells were treated with the vehicle alone (Con), 200 ng/ml lipopolysaccharides (LPS), or 50 or 100 μg/ml OCD20015-V009 and then incubated at 37° C. with 5% CO₂. The time-dependent changes in the mRNA levels of ISG-15, 20, and 56, TNF-α, and IFN-β genes in RAW 264.7 cells after OCD20015-V009 treatment were examined.

Total RNA extraction and cDNA synthesis were conducted using the Easy-BLUE™ RNA extraction kits (iNtRON Biotech) and AccuPower® CycleScript RT PreMix (Bioneer), respectively. A total of 1 μg RNA was reverse-transcribed into cDNA, and qPCR oligonucleotide primers for macrophage cell cDNA are shown in Table 5.

TABLE 5

| SEQ ID NO | Name | Sequence | |
|---|---|---|---|
| 1 | GAPDH | Forward | TGACCACAGTCCATGCCATC |
| 2 | | Reverse | GACGGACACATTGGGGGTAG |
| 3 | ISG-15 | Forward | CAATGGCCTGGGACCTAAA |
| 4 | | Reverse | CTTCTTCAGTTCTGACACCGTCAT |
| 5 | ISG-20 | Forward | AGAGATCACGGACTACAGAA |
| 6 | | Reverse | TCTGTGGACGTGTCATAGAT |
| 7 | ISG-56 | Forward | AGAGAACAGCTACCACCTTT |
| 8 | | Reverse | TGGACCTGCTCTGAGATTCT |
| 9 | TNF-α | Forward | AGCAAACCACCAAGTGGAGGA |
| 10 | | Reverse | GCTGGCACCACTAGTTGGTTGT |
| 11 | IFN-β | Forward | TCCAAGAAAGGACGAACATTCG |
| 12 | | Reverse | TGCGGACATCTCCCACGTCAA |

10 μL of the AccuPower® 2× Greenstar qPCR master mix, 5 μL of template DNA, and 3 μL of RNase-free water. The PCR cycle was as follows: 95° C. for 10 min, 95° C. for 20 s, 60° C. (ISG-15), 53° C. (ISG-20), 60° C. (IFN-β), 56° C. (ISG-56), or 60° C. (TNF-α) for 40 s, and at each experiment end, a melting curve analysis was conducted to confirm that a single product per primer pair was amplified.

Amplification and analysis were performed using the QuantStudio 6 Flex Real-time PCR System (Thermo Fisher), and each sample was compared using the relative CT method. Fold changes in gene expression were determined relative to the blank control after normalization to GAPDH expression using the 2-ΔΔCt method.

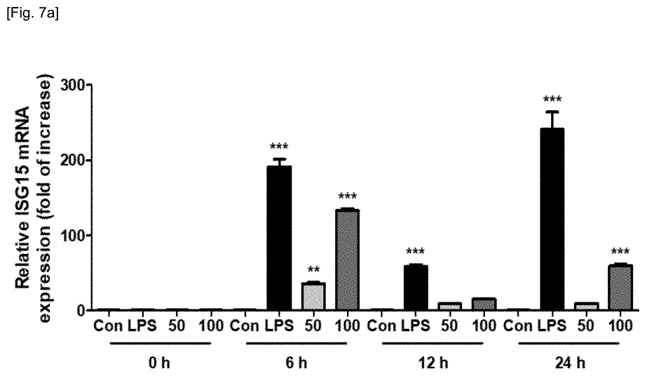

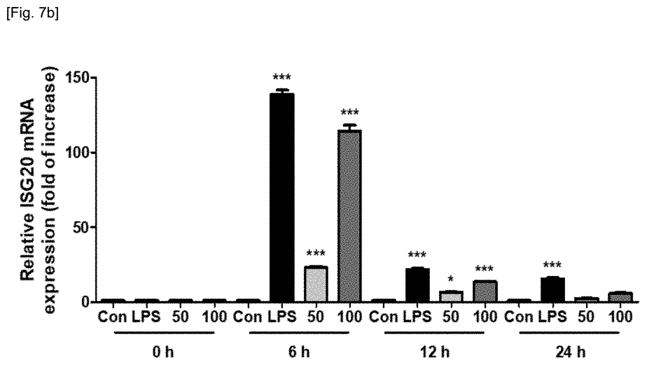

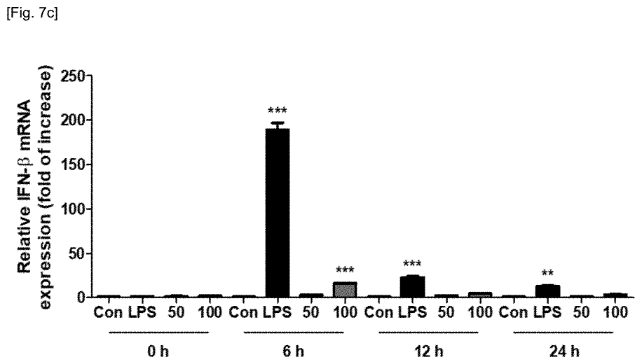

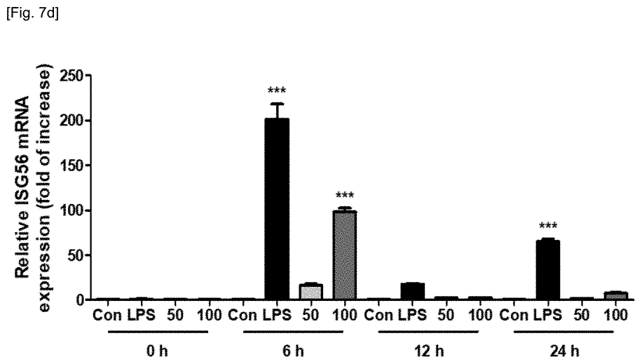

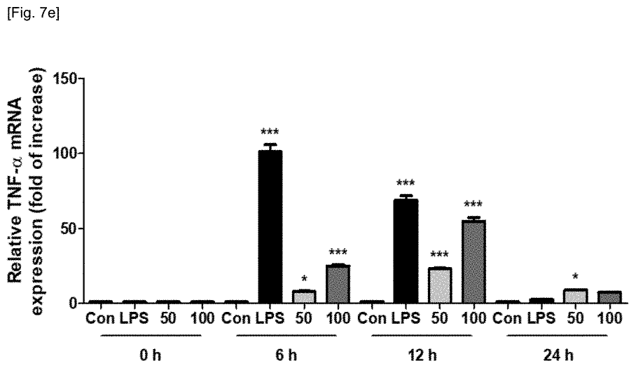

As can be confirmed in FIGS. 7A to 7E, the expression of the IFN-stimulated gene (ISG)-15, ISG-20, and ISG-56, and TNF-α and IFN-β genes increased in the OCD20015-V009-treated RAW 264.7 cells compared with that in the untreated cells, and this pattern was not changed over time. In addition, the observed pattern was similar to that of LPS-treated positive control.

The transcription of the ISG-15, ISG-20, and ISG-56 genes increased by 132.9±5.2, 114.2±6.7, and 98.1±7.5-fold, respectively, in the cells pre-treated with 100 μg/ml OCD20015-V009 for 6 hours. Overall, the results indicate that OCD20015-V009 can induce an antiviral state by modulating the IFN signaling pathway and ISG expression in macrophages, thus inhibiting viral infections.

### Test Example 8: Chemical Composition of OCD20015-V0009 by UPLC-MS/MS Analysis

The components of OCD20015-V009 were identified by analyzing water extracts of each of the traditionally used herbs. The UPLC-MS/MS analysis, which compared the retention time and mass fragmentation of the water extract with the authenticated standards, was performed.

A Dionex UltiMate 3000 system equipped with a Thermo Q-Exactive mass spectrometer (UHPLC-MS/MS, Thermo Fisher Scientific, USA) was used for the phytochemical analysis of OCD20015-V009. Data acquisition and processing were performed using Xcalibur v.3.0 and Tracefinder v.4.0.

Chromatographic separation was achieved with an Acquity BEH C18 column (100 mm×2.1 mm, 1.7 μm, Waters, USA), and the gradient elution consisting of 0.1% formic acid in water and acetonitrile was used. The identified compounds were compared with the retention time and mass spectrum of the authenticated standards. For the constituents that did not match the standards, corresponding m/z and the MS fragment information was found in previous reports. (* standard retention time (Rt) and mass spectrum data comparison)

TABLE 6

| | | | | | | | MS/MS | |
|---|---|---|---|---|---|---|---|---|
| | Rt | Calculated | Estimated | | Error | | Fragments | |
| No | (Min) | (m/z) | (m/z) | Adducts | (ppm) | Formula | (m/z) | Identifications |
| 1 | 1.92 | 407.120 | 407.118 | [M + COOH]⁻ | −3.13 | C₁₅H₂₂O₁₀ | 199, 166 | Catalpol* |
| 2 | 4.51 | 393.140 | 393.139 | [M + COOH]⁻ | −2.64 | C₁₅H₂₄O₉ | 347, 149, 127 | Ajugol* |
| 3 | 4.73 | 375.130 | 375.129 | [M − H]⁻ | −2.44 | C₁₆H₂₄O₁₀ | 213, 169, 113 | Loganic acid* |
| 4 | 5.14 | 353.088 | 353.087 | [M − H]⁻ | −2.62 | C₁₆H₁₈O₉ | 191 | Chlorogenic Acid* |
| 5 | 5.63 | 289.072 | 289.071 | [M − H]⁻ | −2.47 | C₁₅H₁₄O₆ | 289, 245, 205 | Epicatechin* |
| 6 | 6.64 | 447.129 | 447.127 | [M + H]⁺ | −3.96 | C₂₂H₂₂O₁₀ | 283, 268 | Calycosin-7-glucoside* |
| 7 | 6.66 | 463.088 | 463.087 | [M − H]⁻ | −2.51 | C₂₁H₂₀O₁₂ | 301 | Isoguercitrin* |
| 8 | 7.98 | 827.444 | 827.441 | [M − H]⁻ | −2.92 | C₄₂H₆₈O₁₆ | — | Platy saponin A |
| 9 | 8.80 | 991.548 | 991.546 | [M + COOH]⁻ | −2.63 | C₄₈H₈₂O₁₈ | 946, 475, 161 | Ginsenoside Re* |
| 10 | 8.80 | 845.490 | 845.488 | [M + COOH]⁻ | −2.66 | C₄₂H₇₂O₁₄ | 161, 101 | Ginsenoside Rg*1 |
| 11 | 9.58 | 283.061 | 283.060 | [M − H]⁻ | −3.17 | C₁₆H₁₂O₅ | 268 | Calycosin* |
| 12 | 9.94 | 1385.623 | 1385.618 | [M − H]⁻ | −3.80 | C₆₃H₁₀₂O₃₃ | 843, 469 | Platycodin D2* |
| 13 | 10.03 | 1223.570 | 1223.566 | [M − H]⁻ | −3.82 | C₅₇H₉₂O₂₈ | 681, 541, 469 | Platycodin D* |
| 14 | 11.33 | 845.490 | 845.488 | [M + COOH]⁻ | −3.02 | C₄₂H₇₂O₁₄ | 799, 637 | Ginsenoside Rf* |
| 15 | 11.55 | 1153.601 | 1153.598 | [M + COOH]⁻ | −3.13 | C₅₄H₉₂O₂₃ | 1107, 945 | Ginsenoside Rbl* |
| 16 | 11.69 | 815.480 | 815.477 | [M + COOH]⁻ | −2.97 | C₄₁H₇₀O₁₃ | 637, 161 | Notoginsenoside R2* |
| 17 | 11.88 | 1123.591 | 1123.586 | [M + COOH]⁻ | −3.70 | C₅₃H₉₀O₂₂ | 1077, 945 | Ginsenoside Rc* |
| 18 | 12.10 | 955.491 | 955.487 | [M − H]⁻ | −3.59 | C₄₈H₇₆O₁₉ | 955, 793, 523 | GinsenosideRo* |
| 19 | 12.38 | 267.066 | 267.065 | [M − H]⁻ | −3.53 | C₁₆H₁₂O₄ | 252 | Formononetin* |
| 20 | 12.97 | 991.548 | 991.546 | [M + COOH]⁻ | −2.75 | C₄₈H₈₂O₁₈ | 621 | Ginsenoside Rd* |
| 21 | 13.71 | 871.470 | 871.467 | [M + COOH]⁻ | −2.81 | C₄₃H₇₀O₁₅ | — | Astragaloside II |
| 22 | 14.36 | 871.470 | 871.467 | [M + COOH]⁻ | −3.02 | C₄₃H₇₀O₁₅ | — | Isoastragalosides II |
| 23 | 14.96 | 871.470 | 871.467 | [M + COOH]⁻ | −3.16 | C₄₃H₇₀O₁₅ | — | Astragaloside II isomer |
| 24 | 15.60 | 913.480 | 913.478 | [M + COOH]⁻ | −2.96 | C₄₅H₇₂O₁₆ | — | Astragaloside I |
| 25 | 16.11 | 497.327 | 497.326 | [M − H]⁻ | −2.72 | C₃₁H₄₆O₅ | 419, 405, 403 | 6α-Hydroxypolyporenic |
| | | | | | | | | acid C* |
| 26 | 16.20 | 913.480 | 913.477 | [M + COOH]⁻ | −3.09 | C₄₅H₇₂O₁₆ | — | Isoastragaloside I |
| 27 | 16.55 | 767.494 | 767.490 | [M + H]⁺ | −4.67 | C₄₂H₇₀O₁₂ | 443, 425, 407 | Ginsenoside Rg5* |
| 28 | 16.84 | 913.480 | 913.477 | [M + COOH]⁻ | −3.16 | C₄₅H₇₂O₁₆ | — | Astragaloside I isomer |
| 29 | 18.19 | 483.348 | 483.346 | [M − H]⁻ | −3.32 | C₃₁H₄₈O₄ | 437, 423, 405, 389 | Dehydrotumulosic acid* |
| 30 | 18.33 | 811.485 | 811.482 | [M + COOH]⁻ | −3.17 | C₄₂H₇₀O₁₂ | 765, 603 | Ginsenoside Rk2* |
| 31 | 18.57 | 497.327 | 497.326 | [M − H]⁻ | −3.03 | C₃₁H₄₆O₅ | 423, 379, 211 | Poricoic acid A* |
| 32 | 18.94 | 481.332 | 481.331 | [M − H]⁻ | −3.22 | C₃₁H₄₆O₄ | 435, 421, 311 | Polyporenic acid C* |
| 33 | 19.22 | 483.348 | 483.346 | [M − H]⁻ | −3.20 | C₃₁H₄₈O₄ | 437, 423, 337 | 3-Epidehydrotumulosic |
| | | | | | | | | acid* |
| 34 | 20.31 | 525.359 | 525.357 | [M − H]⁻ | −3.08 | C₃₃H₅₀O₅ | 465, 355 | Dehydropachymic acid* |
| 35 | 20.54 | 527.374 | 527.373 | [M − H]⁻ | −2.88 | C₃₃H₅₂O₅ | 527, 405 | Pachymic acid* |

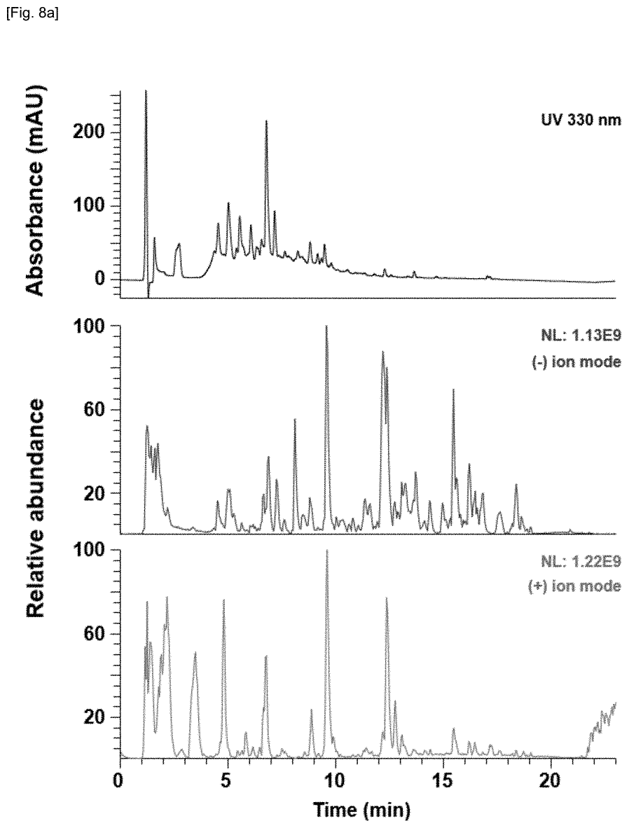

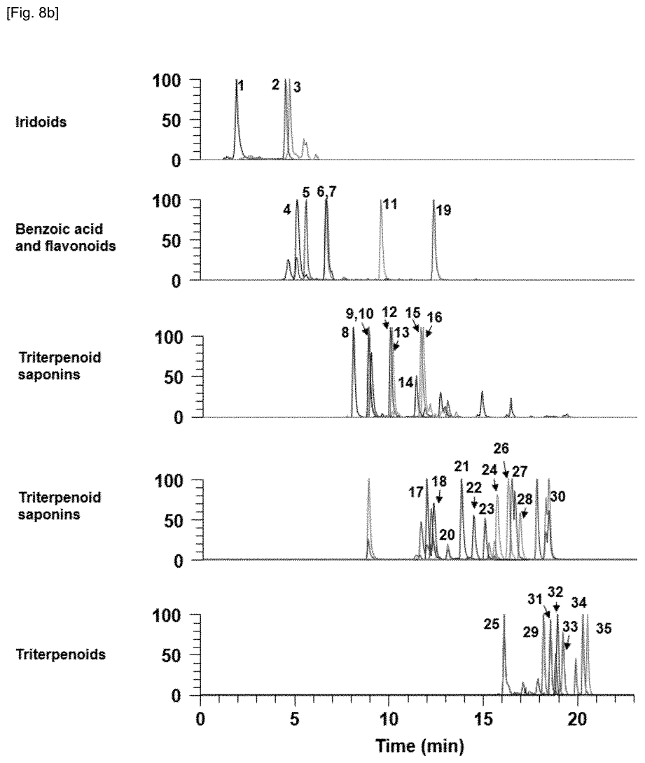

As can be confirmed in Table 6 and FIG. 8, the results revealed components including one type of benzoic acid (chlorogenic acid), three types of iridoids (catalpol, ajugol, and loganic acid), five types of flavonoids (epicatechin, calycosin-7-glucoside, isoquercitrin, calycosin, and formononetin), seven types of triterpenoids (6α-hydroxypolyporenic acid C, dehydrotumulosic acid, poricoic acid A, polyporenic acid C, 3-epidemic acid, dehydropachymic acid, and pachymic acid), and 19 types of triterpenoid saponins (platy saponin A, ginsenoside Re, ginsenoside Rg1, platycodin D2, platycodin D, ginsenoside Rf, ginsenoside Rb1, notoginsenoside R2, ginsenoside Rc, astragaloside II isomer, astragaloside I, isoastragaloside I, ginsenoside Rg5, astragaloside I isomer, and ginsenoside Rk2).

The antiviral effect of OCD20015-V009 on IAV may be attributed to the effects of these compounds. Next to the viral replication inhibitory effects of the components identified in OCD20015-V009, it was investigated whether eight major compounds of OCD20015-V009, that is, chlorogenic acid, ginsenoside Rg1, calycosin, ginsenoside Rb1, ginsenoside Rd, astragaloside II, astragaloside I, and polygalacin D, inhibited the influenza virus replication in RAW 264.7 cells by suppressing the production of the viral proteins.

Specifically, 12 hours after the treatment with RAW 264.7 cells alone, 10 μM eight types of compounds derived from OCD20015-V009, or 1000 U/mL recombinant mouse interferon-β, RAW 264.7 cells were infected with GFP-expressing influenza virus A/PR/8/34-GFP at the multiplicity of infection of 10 μM, and 24 hours after viral infection, the GFP levels and reduction in viral replication were analyzed through the images obtained using flow cytometry.

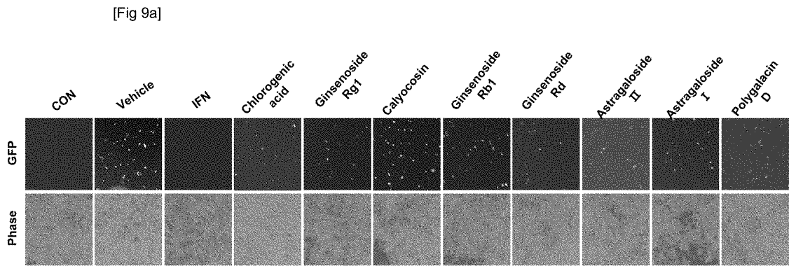

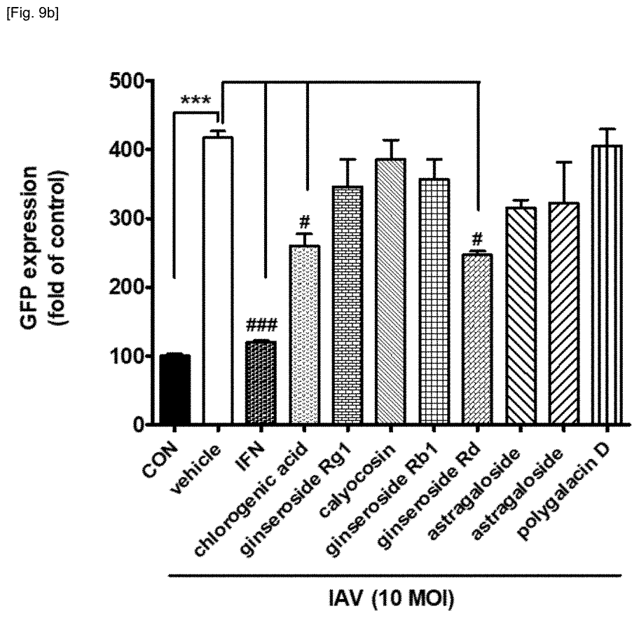

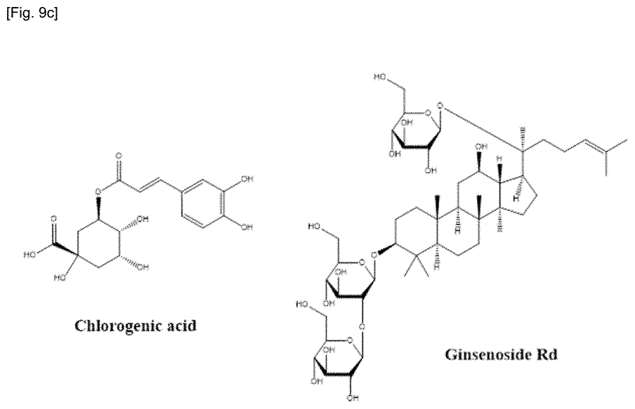

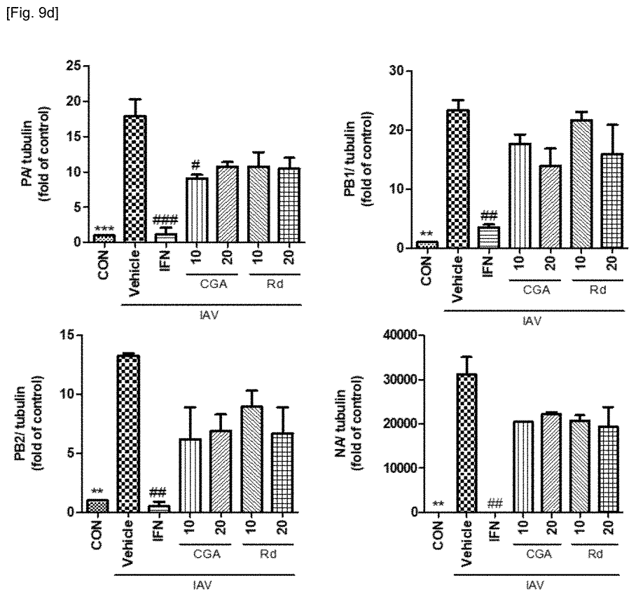

As can be confirmed in FIGS. 9A and 9B, the level of GFP was lower in cells pre-treated with chlorogenic acid or ginsenoside Rd than that in the untreated cells.

The levels of the IAV proteins in RAW 264.7 cells as assayed by Western blots were analyzed using antibodies against various IAV proteins. RAW 264.7 cells were pre-treated with 10 and 20 μM chlorogenic acid or ginsenoside Rd, 1000 U/mL recombinant mouse interferon (IFN)-β, or the medium only (negative control) after viral adsorption.

Specifically, the levels of IAV proteins PA, PB1, PB2, and NA in the cell lysates were analyzed with Western blots, and the level of tubulin was used as an internal control. Western blotting of viral expressions was performed and data were quantified using the ImageJ software.

As can be confirmed in FIG. 9D, the Western blots showed that the levels of IAV proteins were suppressed in the RAW 264.7 cells pre-treated with chlorogenic acid or ginsenoside Rd compared with those in the untreated cells.

What is claimed is:

1. A method for enhancement of innate immunity, antiviral use, or alleviation, prevention, or treatment of a viral infection, the method comprising:
administering to a subject a composition containing a first complex extract of Codonopsis and Rehmannia.

2. The method of claim 1, wherein the first complex extract contains 5.9-8.9 wt % of Codonopsis and 74.1-94.1 wt % of Rehmannia.

3. The method of claim 1, wherein the first complex extract further contains at least one selected from the group consisting of Ginseng and Platycodon.

4. The method of claim 3, wherein the first complex extract contains 5.4-8.2 wt % of Codonopsis, 6.4-9.6 wt % of Ginseng, and 68.2-88.2 wt % of Rehmannia.

5. The method of claim 3, wherein the first complex extract contains 5.0-7.4 wt % of Codonopsis, 62.0-82.0 wt % of Rehmannia, and 13.0-19.6 wt % of Platycodon.

6. The method of claim 3, wherein the first complex extract further contains at least one selected from the group consisting of Poria and Hawthorn fruit.

7. The method of claim 6, wherein the first complex extract contains 4.0-6.0 wt % of Codonopsis, 4.7-7.1 wt % of Ginseng, 50.2-70.2 wt % of Rehmannia, 9.7-14.5 wt % of Poria, 0.9-1.3 wt % of Hawthorn fruit, and 10.6-15.8 wt % of Platycodon.

8. The method of claim 1, wherein the composition exhibits antiviral activity against at least one selected from the group consisting of influenza virus, corona virus, vesicular stomatitis virus, and Newcastle disease virus, and activity to alleviate, prevent, or treat a viral infection caused by the at least one selected from the group.

9. A method for enhancement of innate immunity, antiviral use, or alleviation, prevention, or treatment of a viral infection, the method comprising:
administering to a subject a composition containing a second complex extract of two or more selected from the group consisting of Codonopsis, Rehmannia, Ginseng, Platycodon, Poria, Hawthorn fruit, and Astragalus.

10. The method of claim 9, wherein the second complex extract contains 1.25-5.0 wt % of Codonopsis, 12.50-50.0 wt % of Rehmannia, 1.25-5.00 wt % of Ginseng, 6.85-27.0 wt % of Platycodon, 2.50-10.0 wt % of Poria, 0.65-2.50 wt % of Hawthorn fruit, and 25.0-80.0 wt % of Astragalus.

11. The method of claim 9, wherein the second complex extract contains at least one compound selected from the group consisting of chlorogenic acid, ginsenoside Rg1, calycosin, ginsenoside Rb1, ginsenoside Rd, astragaloside II, astragaloside I, and polygalacin D.

12. The method of claim 9, wherein the composition exhibits antiviral activity against at least one selected from the group consisting of influenza virus, corona virus, vesicular stomatitis virus, and Newcastle disease virus, and activity to alleviate, prevent, or treat a viral infection caused by the at least one selected from the group.


SEQUENCE LISTING (12 sequences)

SEQ ID NO: 1 - DNA, 20 bp, Artificial Sequence
  Synthetic Construct
tgaccacagtccatgccatc

SEQ ID NO: 2 - DNA, 20 bp, Artificial Sequence
  Synthetic Construct
gacggacacattgggggtag

SEQ ID NO: 3 - DNA, 19 bp, Artificial Sequence
  Synthetic Construct
caatggcctgggacctaaa

SEQ ID NO: 4 - DNA, 24 bp, Artificial Sequence
  Synthetic Construct
cttcttcagttctgacaccgtcat

SEQ ID NO: 5 - DNA, 20 bp, Artificial Sequence
  Synthetic Construct
agagatcacggactacagaa

SEQ ID NO: 6 - DNA, 20 bp, Artificial Sequence
  Synthetic Construct
tctgtggacgtgtcatagat

SEQ ID NO: 7 - DNA, 20 bp, Artificial Sequence
  Synthetic Construct
agagaacagctaccaccttt

SEQ ID NO: 8 - DNA, 20 bp, Artificial Sequence
  Synthetic Construct
tggacctgctctgagattct

SEQ ID NO: 9 - DNA, 21 bp, Artificial Sequence
  Synthetic Construct
agcaaaccaccaagtggagga

SEQ ID NO: 10 - DNA, 22 bp, Artificial Sequence
  Synthetic Construct
gctggcaccactagttggttgt

SEQ ID NO: 11 - DNA, 22 bp, Artificial Sequence
  Synthetic Construct
tccaagaaaggacgaacattcg

SEQ ID NO: 12 - DNA, 21 bp, Artificial Sequence
  Synthetic Construct
tgcggacatctcccacgtcaa